(19) United States
(12) Patent Application Publication
(10) Pub. No.: US 2023/0124467 A1
(43) Pub. Date: Apr. 20, 2023

(54) COMPOUNDS AND METHODS FOR TREATING DISEASES AND/OR CONDITIONS CAUSED BY CORONAVIRUS

(71) Applicant: Mios Pharmaceuticals Limited, Hong Kong (HK)

(72) Inventors: Wai Yip Thomas LEE, Hong Kong (HK); Chung Sing Daniel POON, Hong Kong (HK)

(21) Appl. No.: 17/908,924

(22) PCT Filed: Mar. 3, 2021
(86) PCT No.: PCT/CN2021/078875

§ 371 Date: Sep. 2, 2022

Related U.S. Application Data
(60) Provisional application No. 62/984,771, filed on Mar. 3, 2020.

(51) Int. Cl. A61K 31/53; A61K 31/706; A61P 31/14

(52) U.S. Cl. CPC A61K 31/53; A61K 31/706; A61P 31/14

(57) ABSTRACT

Compounds and compositions, for example vardenafil, for treating diseases and/or conditions caused by, arising from, and/or associated with coronavirus in a subject, for example a subject infected with SARS-CoV-2 or other coronavirus. Combinations of agents useful for treating diseases and/or conditions caused by, arising from, and/or associated with coronavirus in a subject, for example, vardenafil and Remdesivir, are also provided.

### 1. CROSS REFERENCE TO RELATED APPLICATIONS

This application claims the priority benefit of U.S. Provisional Application No. 62/984,771, filed Mar. 3, 2020, the contents of which are incorporated herein in their entireties by reference thereto.

### 2. BACKGROUND

The coronavirus SARS-CoV-2 causes coronavirus disease 2019 (COVID-19). Symptoms of COVID-19 include fever or chills, cough, shortness of breath or difficulty breathing, fatigue, muscle or body aches, headache, loss of taste or smell, sore throat, congestion or runny nose, nausea or vomiting, and diarrhea (cdc.gov/coronavirus/2019-ncov/symptoms-testing/symptoms.html). Severe cases of COVID-19 can be fatal. To date, there have been over 100 million confirmed cases of COVID-19 worldwide, with over 2.4 million deaths reported to the World Health Organization (WHO) due to COVID-19 (WHO Coronavirus Disease (COVID-19) Dashboard (covid19.who.int)).

While multiple SARS-CoV-2 vaccines have been developed, less than 3% of the world’s population has been vaccinated as of mid-February 2021 (ourworldindata.org/covid-vaccinations). Thus, COVID-19 remains a global health threat.

Beyond SARS-CoV-2, numerous other coronaviruses can cause illness in humans and animals. For example, SARS-CoV emerged in 2002 and caused severe acute respiratory syndrome (SARS), while MERS-CoV causes Middle East respiratory syndrome (MERS) (niaid.nih.gov/diseases-conditions/coronaviruses).

New treatments for diseases and conditions caused by coronaviruses, such as COVID-19, are needed.

### 3. SUMMARY

The present disclosure provides compounds and compositions for treating diseases and/or conditions caused by, arising from, and/or associated with coronavirus in a subject.

In one aspect, the disclosure provides vardenafil, a phosphodiesterase type 5 (PDE5) inhibitor, for use in the treatment of a coronavirus infection in a subject, e.g., a subject infected with SARS-CoV-2 or other coronavirus. Vardenafil can be administered as monotherapy or in combination with one or more additional agents, for example an antiviral agent such as Remdesivir. The inventors initially identified vardenafil as being useful for treating a coronavirus infection by a computerized screen. Vardenafil’s activity against SARS-CoV-2 was validated by in vitro studies, described herein in Example 1. It was then surprisingly discovered that vardenafil and Remdesivir have synergistic antiviral effects when used in combination, as described herein in Example 2. Thus, the disclosure is based, in part, on the surprising discovery that the antiviral activity of vardenafil can be synergistically enhanced when used in combination with the antiviral medication Remdesivir.

In various aspects, the disclosure provides uses of vardenafil in the manufacture of a medicament for treating a coronavirus infection, uses of antiviral agents such as Remdesivir in the manufacture of a medicament for treating coronavirus infection, vardenafil for use in the treatment of a coronavirus infection, antiviral medications such as Remdesivir for use in combination with vardenafil, combinations of vardenafil and additional agents, e.g., Remdesivir, and methods of treating subjects having or suspected of having a coronavirus infection with vardenafil.

In other aspects, the disclosure provides compounds and combinations of compounds for treating diseases and/or conditions caused by, arising from, and/or associated with coronavirus in a subject, wherein the compounds and combinations of compounds comprise lopinavir, ritonavir, atazanavir, indinavir sulfate, nelfinavir mesylate, ceftaroline fosamil, leucal (also known as leucovorin), aztreonam, cangrelor, vardenafil, fludarabine, eltrombopag, tedizolid, macitentan, cobicistat, and/or lifitegrast, and any derivatives thereof. In some embodiments, the compound is vardenafil. In some embodiments, a combination of compounds comprises vardenafil.

Vardenafil, uses of vardenafil, combinations comprising vardenafil, and methods of using vardenafil are further described in Section 5.1 and numbered embodiments 1 to 253, in Section 7.2, infra.

Exemplary additional agents that can be used for treating a coronavirus infection, for example in combination with vardenafil, are described in Section 5.2 and numbered embodiments 3 to 27 and 30 to 68 and 84 to 100 in Section 7.2, infra.

Vardenafil and other compounds described herein can be administered in pharmaceutical compositions. Exemplary features of pharmaceutical compositions are described in Section 5.3 and numbered embodiments 27, 32 to 34, 65 to 68, 81, 82, 87, and 91, in Section 7.2, infra.

Features of exemplary coronaviruses treatable with the compounds, compositions, and methods of the disclosure are described in Section 5.4 and numbered embodiments 111 to 245 in Section 7.2, infra.

Features of exemplary subject populations treatable with the compounds, compositions of the disclosure are described in Section 5.5 and numbered embodiments 103 to 110 in Section 7.2, infra.

### 4. BRIEF DESCRIPTION OF THE DRAWINGS

FIG. 1A.1-1B.2 show antiviral effects of the 14 compounds in preliminary antiviral activity screen (Example 1). FIGS. 1A.1-1A.2: plates with 20 PFU/well of SARS-CoV-2; FIGS. 1B.1-1B.2: no virus control plates. Compound identity is as shown in Table 2.

FIGS. 2A.1-2E.2 show antiviral effects of the 14 compounds against 20 PFU/ well SARS-CoV-2 in VeroE6 cells (Example 1). FIG. 2A.1-2A.2: images of the front and back of the plate for compounds 1 to 3, respectively; FIGS. 2B.1-2B.2: images of the front and back of the plate for compounds 4 to 6, respectively; FIGS. 2C.1-2C.2: images of the front and back of the plate for compounds 7 to 9, respectively; FIGS. 2D.1-2D.2: images of the front and back of the plate for compounds 10 to 12, respectively; FIGS. 2E.1-2E.2: images of the front and back of the plate for compounds 13 and 14, respectively. Compound identity is as shown in Table 2.

FIG. 3A-3B.2 show antiviral effects of compounds 3, 6 and 13 alone at different drug concentrations (Example 1). FIG. 3A: images of the front (left panel) and back (right panel) of the plate for compounds 6 alone and 13 alone; FIGS. 3B.1-3B.2: images of the front and back of the plate for compound 3 alone and controls, respectively. Compound identity is as shown in Table 2.

FIGS. 4A-4C show cytotoxicity of compounds in an MTT assay (Example 1). FIG. 4A: compounds 2, 3, 8, 9, 12, 13, and 14; FIG. 4B: compounds 1, 6, 7, 10, 11, 4, and 5; FIG. 4C: compounds 3, 6, and 13. Compound identity is as shown in Table 2.

FIGS. 5A-5B are schematics showing the preparation of pairwise combinations of vardenafil (compound 13), nelfinavir (compound 3), and hydroxychloroquine (compound 6), and Remdesivir (R) (i.e., combinations 3+13, 3+6, 6+13, 3+R, 6+R, 13+R) (FIG. 5A) and triple combinations of drugs evaluated in Example 2 (i.e., combinations 3+6+13 (FIG. 5B)).

FIG. 6A.1-6B.2 show antiviral effects of pairwise combinations of vardenafil (compound 13), nelfinavir (compound 3), and hydroxychloroquine (compound 6) (i.e., combinations 3+13, 3+6, 6+13 (Example 2). FIGS. 6A.1-6A.2: images of the front and back of the no virus control plate, respectively; FIGS. 6B.1-6B.2: images of the front and back of the plate with SARS-CoV-2, respectively. Drug concentrations used in the plates are shown in Table 3B.

FIGS. 7A-7B show antiviral effect of Remdesivir alone (Example 2). FIG. 7A: an image of the front of plate; FIG. 7B: an image of the back of plate. Drug concentrations of Remdesivir used in the plate are shown in Table 3C.

FIG. 8A.1-8B.2 show antiviral effects of pairwise combinations of Remdesivir (R) with vardenafil (compound 13), nelfinavir (compound 3), and hydroxychloroquine (compound 6) (i.e., combinations 3+R, 6+R, 13+R and R only) (Example 2). FIGS. 8A.1-8A.2: images of the front and back of the control plate with no virus, respectively; FIGS. 8B.1-8B.2: images of the front and back of the plate with SARS-CoV-2, respectively. Drug concentrations used in the plates are shown in Table 3D.

FIGS. 9A-9B show antiviral effects of triple combinations of vardenafil (compound 13), nelfinavir (compound 3), and hydroxychloroquine (compound 6) (i.e., combination 3+6+13) (Example 2). FIG. 9A: no virus control plate; FIG. 9B, plate with SARS-CoV-2. Drug concentrations used in the plates are shown in Table 3E. In FIGS. 9A-9B, the left panel is an image of the front of the plate, and the right panel is an image of the back of the plate.

### 5. DETAILED DESCRIPTION

The present disclosure provides compounds and compositions for treating diseases and/or conditions caused by, arising from, and/or associated with coronavirus in a subject.

The diseases and/or conditions can comprise, for example, one or more of severe acute respiratory syndrome (SARS), Middle East respiratory syndrome (MERS), Coronavirus disease 2019 (COVID-19), pulmonary infection, respiratory tract infections, cold, fever, upper and/or lower respiratory tract infections and/or diseases, pneumonia, diarrhea, dry cough, dyspnea, sore throat, any other infections. In some embodiments, the disease and/or condition is COVID-19.

A compound or composition of the disclosure in some embodiments targets one or more proteins of a coronavirus, including but not limited to 3CL-protease and/or RNA-dependent RNA polymerase (RdRP, RDR, also known as RNA replicase).

A compound or composition of the disclosure can in some embodiments target coronavirus in general without any specific protein(s) thereof.

In some aspects, the compound or combination can be one or more of: lopinavir, ritonavir, atazanavir, indinavir sulfate, nelfinavir mesylate, ceftaroline fosamil, leucal (also known as leucovorin), aztreonam, cangrelor, vardenafil, fludarabine, eltrombopag, tedizolid, macitentan, cobicistat, and/or lifitegrast, and any derivatives thereof. The foregoing compounds were identified as useful for treating a coronavirus infection, particularly SARS-CoV-2, by a computerized model. In some embodiments, the compound is nelfinavir mesylate. In some embodiments, the compound is vardenafil. In some embodiments, a combination comprises vardenafil and nelfinavir mesylate.

In some aspects, the disclosure provides vardenafil for use in combination with Remdesivir.

In some aspects, the present disclosure provides a method of treating a coronavirus infection, diseases and/or conditions caused by, arising from, and/or associated with coronaviruses comprising administering a compound described herein or a composition comprising the compound to a subject in need thereof. For example, the methods of the disclosure in some embodiments comprise administering an amount of vardenafil that is effective (as monotherapy or in combination with an additional agent such as Remdesivir) to reduce one or more symptoms of a coronavirus infection and/or reduce the duration of the coronavirus infection. Exemplary symptoms of a coronavirus infection, e.g., symptoms of COVID-19, include fever or chills, cough, shortness of breath or difficulty breathing, fatigue, muscle or body aches, headache, loss of taste or smell, sore throat, congestion or runny nose, nausea or vomiting, and diarrhea.

### 5.1. Vardenafil

Vardenafil (IUPAC name 2-[2-ethoxy-5-(4-ethylpiperazin-1-yl)sulfonylphenyl]-5-methyl-7-propyl-3H-imidazo[5,1-f][1,2,4]triazin-4-one) is a phosphodiesterase type 5 (PDE5) inhibitor marketed for the treatment of erectile dysfunction under the trade names Levitra®, Staxyn™, and Vivanza™. Marketed dosage forms include 2.5 mg, 5 mg, 10 mg, and 20 mg tablets for oral use. Marketed forms of vardenafil typically comprise vardenafil as vardenafil hydrochloride. Medicaments comprising vardenafil in the form of vardenafil hydrochloride trihydrate have also been described, for example in U.S. 8,841,446 and 8,273,876, the contents of which are incorporated herein by reference in their entireties. Thus, vardenafil can be used in the methods and compositions of the disclosure, for example, in the form of 2-[2-ethoxy-5-(4-ethylpiperazin-1-yl)sulfonylphenyl]-5-methyl-7-propyl-3H-imidazo[5,1-f][1,2,4]triazin-4-one, a pharmaceutically acceptable salt thereof, or a solvate (e.g., hydrate) of any of the foregoing. Exemplary pharmaceutically acceptable salts include, but are not limited to ammonium salts, hydrochlorides, carbonates, bicarbonates, acetates, lactates, butyrates, propionates, sulfates, methanesulfonates, citrates, tartrates, nitrates, sulfonates, oxalates and /or succinates.

In some embodiments, vardenafil is in a form which is amorphous, e.g., as described in U.S. application publication 2007/0197535, the contents of which are incorporated herein by reference in their entireties. In other embodiments, vardenafil is in a crystalline form, e.g., as described in U.S. application publication 2007/0197535. In some embodiments, the vardenafil is in the form of vardenafil hydrochloride. In some embodiments, the vardenafil is in the form of vardenafil hydrochloride trihydrate. Various vardenafil dosage forms are described in the art, for example, in US Patent Nos. 8,273,876, 8,841,446, and 8,613,950, the contents of which are incorporated herein by reference in their entireties, and such dosage forms can be used in the methods and compositions of the disclosure.

The methods of the disclosure can comprise administering an amount of vardenafil to a subject effective to reduce the severity of one or more symptoms of a coronavirus infection and/or effective to reduce the duration of one or more symptoms of a coronavirus infection, e.g., fever or chills, cough, shortness of breath or difficulty breathing, fatigue, muscle or body aches, headache, loss of taste or smell, sore throat, congestion or runny nose, nausea or vomiting, diarrhea, or a combination thereof.

Actual dosage levels of vardenafil and other compounds disclosed herein can be an amount which is effective to achieve the desired therapeutic response for a particular subject, composition, and mode of administration, preferably without being toxic to the subject. The selected dosage level can depend upon a variety of pharmacokinetic factors including the route of administration, the time of administration, the duration of the treatment, other agents (e.g., additional active agents such as those described in Section 5.2 or compounds and/or inert materials used as carriers) used in combination with an active agent, the age, sex, weight, condition, general health and prior medical history of the subject being treated, and like factors known in the medical arts.

### 5.2. Additional Agents

In some aspects, vardenafil can be used in combination with one or more additional agents. For example, vardenafil can be used in combination with one or more antiviral agents, e.g., Remdesivir or another antiviral agent described herein. In some embodiments, the additional agent is an antiviral agent that targets RNA-dependent RNA polymerase (RdRP). Exemplary antiviral agents that be used include protease inhibitors, for example, amprenavir, atazanavir, indinavir, darunavir, fosamprenavir, nelfinavir, ritonavir, saquinavir, tipranavir or lopinavir; nucleoside analogs, for example, Remdesivir, ribavirin, didanosine, vidarabine, galidesivir, Remdesivir, emtricitabine, lamivudine, zalcitabine, abacavir, acyclovir, entecavir, stavudine, telbivudine, zidovudine, idoxuridine, or trifluridine; and interferons, for example, interferon beta 1b. Combinations of antiviral agents can also be used.

In some embodiments, an additional agent comprises lopinavir, ritonavir, atazanavir, indinavir sulfate, nelfinavir mesylate, ceftaroline fosamil, leucal (also known as leucovorin), aztreonam, cangrelor, fludarabine, eltrombopag, tedizolid, macitentan, cobicistat, or lifitegrast, or a combination thereof.

In some embodiments, an additional agent in Remdesivir. Remdesivir, marketed as Veklury® (Gilead), is an antiviral medication that targets RdRP and has been approved by the US FDA for treating COVID-19 (www.gilead.com/news-and-press/press-room/press-releases/2020/10/us-food-and-drug-administration-approves-gileads-antiviral-veklury-remdesivir-for-treatment-of-covid19; Ju FA, 2020, J Nanomedine Biotherapeutic Discov 10:164. doi: 10.35248/2155-983X.10.164). Remdesivir is available in two dosage forms: a lyophilized powder which can be reconstituted before administration as an infusion, and as a 5 mg/ml solution which can be diluted prior to administration as an infusion. The Veklury® prescribing information indicates that the recommended dosage of Remdesivir for treating COVID-19 in adult and pediatric subjects 12 years of age an older and weighing at least 40 kg is a single loading dose of 200 mg on day 1 via IV infusion followed by once-daily maintenance doses of 100 mg from day 2 via IV infusion. The Veklury® prescribing information further recommends that the duration of treatment be 5 days, which can be extended up to 5 additional days. Accordingly, in some embodiments, Remdesivir is administered to a subject in an initial loading dose on day 1 of 200 mg, followed by once-daily maintenance doses of 100 mg for 5 to 10 days (e.g., 5 days or 10 days).

In some embodiments, the methods of the disclosure comprise administering to a subject an amount of vardenafil and an amount of Remdesivir that together in combination are therapeutically effective to treat the subject, e.g., effective to reduce the severity and/or duration of one or more symptoms of the coronavirus infection.

Administered “in combination,” as used herein, means that two (or more) different treatments are delivered to the subject during the course of the subject’s affliction with a coronavirus, e.g., the two or more treatments are delivered after the subject has been diagnosed with a coronavirus infection and before the symptoms of the coronavirus infection have subsided. In some embodiments, the delivery of one treatment is still occurring when the delivery of the second begins, so that there is overlap in terms of administration. This is sometimes referred to herein as “simultaneous” or “concurrent delivery.” The term “concurrently” is not limited to the administration of therapies (e.g., vardenafil and an additional agent) at exactly the same time, but rather it is meant that a pharmaceutical composition comprising a first agent is administered to a subject in a sequence and within a time interval such that the first agent can act together with the additional therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic effect. Each therapy can be administered to a subject separately, in any appropriate form and by any suitable route. For example, in some embodiments vardenafil can be administered orally, while Remdesivir can be administered by IV infusion. Alternatively, agents administered “in combination” can be administered to a subject in the same pharmaceutical composition, for example administration of vardenafil in combination with Remdesivir can comprise administration of a single pharmaceutical composition comprising both Remdesivir and vardenafil.

### 5.3. Pharmaceutical Compositions and Mode of Administration

Compound and compositions of the present disclosure, e.g., vardenafil and the other agents described herein, such as Remdesivir, can be formulated into one or more of the following forms: solution, tablet, capsule, granules, powders, crystals, gel, liquid, solvent, paste, pellet, or any other suitable forms.

A pharmaceutical composition can comprise a single active agent, for example, vardenafil, or can comprise more than one active agent, for example both vardenafil and Remdesivir.

A compound or composition of the present disclosure can be administered to a subject via oral, intraperitoneal, intramuscular, intravenous, inhalation, topical, buccal, injection, nasal, transdermal, intradermal, translingual, urethral, vaginal, enteral, rectal, ophthalmic, subcutaneous, irrigation, mouth, throat, transmucosal, intravitreal, otic, perfusion, perivascular, and/or sublingual, or any other suitable routes of administration.

A pharmaceutical composition may additionally comprise one or more of pharmaceutically acceptable carrier, ester, salt, acid, alkali, vehicle, excipient, binder, agent, diluent, stabilizer, emulsifier, absorbent, surfactant, , lubricant, disintegrant, glidant, buffer, filler, gels, preservative, dissolution, liquid, solvent, medium, and/or any other suitable materials / complex.

### 5.4. Coronavirus

Various types of coronavirus are known to infect and cause disease in humans and animals. See, e.g., Desforges et al., 2000, Viruses 12(1):14; (www.nfid.org/infectious-diseases/coronaviruses). Coronaviruses primarily infect the upper respiratory or gastrointestinal tract of mammals and birds. Coronaviruses that can infect humans include SARS-CoV, which causes severe acute respiratory syndrome (SARS), SARS-CoV-2, which causes COVID-19, and Middle East respiratory syndrome coronavirus (MERS-CoV), which causes a lower respiratory tract infection in humans. Coronaviruses, for example Human coronavirus 229E, are believed to cause a significant percentage of common colds in humans. Coronaviruses also cause a range of diseases in livestock animals and domesticated pets, some of which can be serious and are a threat to the farming industry. Economically significant coronaviruses of livestock animals include infectious bronchitis virus (IBV), which mainly causes respiratory disease in chickens and seriously affects the poultry industry worldwide; porcine coronavirus (transmissible gastroenteritis, TGE) and bovine coronavirus, which both result in diarrhea in young animals. Feline coronavirus has at least two forms. Feline enteric coronavirus is a pathogen of minor clinical significance, but spontaneous mutation of this virus can result in feline infectious peritonitis (FIP), a disease associated with high mortality. There are also at least two types of canine coronavirus (CCoV), one that causes mild gastrointestinal disease and one that has been found to cause respiratory disease. Mouse hepatitis virus (MHV) is a coronavirus that causes an epidemic murine illness with high mortality, especially among colonies of laboratory mice.

Compounds and compositions described herein, e.g., the agents described in Sections 5.1 and 5.2, can be used to treat such coronaviruses. In some embodiments, the virus is SARS-CoV-2, which causes COVID-19. In some embodiments, the SARS-CoV-2 is a SARS-CoV-2 variant. Exemplary SARS-CoV-2 variants are described in Section 5.4.1. In other embodiments, the virus is a coronavirus other than SARS-CoV-2, for example, one of the coronaviruses described in this Section or in Section 5.4.2.

### 5.4.1. SARS-CoV-2

In various aspects of the disclosure, the compounds described herein, for example vardenafil, can be used to treat a SARS-CoV-2 infection. There are several known variants of SARS-CoV-2, including variant B.1.1.7 that was detected in the United Kingdom, variant B.1.351 that was detected in South Africa, variant P.1 that was detected in Brazil, variant P.2 that was detected in Brazil, called B.1.1.207 that was detected in Nigeria, variant B.1.525 that was detected in the United Kingdom, and a variant called Cluster 5 that was detected in
Denmark. See, e.g., Wang et al., 2021, Communications Biology 4:228 (doi.org/10.1038/s42003-021-01754-6); www.cdc.gov/coronavirus/2019-ncov/more/science-and-research/scientific-brief-emerging-variants.html;
www.cdc.gov/mmwr/volumes/70/wr/mm7003e2.htm;
www.nytimes.com/interactive/2021/health/coronavirus-variant-tracker.html.

In some embodiments, the SARS-CoV-2 is a SARS-CoV-2 variant, for example, B.1.1.7, B.1.351, P.1, P.2, B. 1.207, B. 1.525, Cluster 5, or a variant sharing one or more mutations with one or more of the foregoing variants. For example, a SARS-CoV-2 variant can have one or more of the mutations set forth in Table 1.

TABLE 1

Exemplary mutations in SARS-CoV-2 variants

| | | |
|---|---|---|
| Amino Acid Mutation (exemplary corresponding nucleotide mutation) | Protein | Exemplary variants having mutations |
| 69-70del | spike protein | B.1.1.7; Cluster 5 |
| K417N | spike protein | B.1.351; P.1 |
| L452R | spike protein | |
| Y453F | spike protein | Cluster 5 |
| E484K | spike protein | B.1.351; P.1; B.1.525; P.1; P.2 |
| N501Y | spike protein | B.1.1.7; B.1.351; P.1 |
| D614G | spike protein | B.1.1.7 |
| P681H | spike protein | B.1.2.207; B.1.1.7 |
| F888L | spike protein | B.1.525 |
| D614G (23403A>G) | spike protein | |
| Q677P | spike protein | |
| Q677H | spike protein | |
| P323L (14408C>T) | NSP12 (RdRP) | |
| Q57H (25563G>T) | ORF3a | |
| T85I (1059C>T) | NSP2 | |
| R203K (28881G>A or 28882G>A) | nucleocapsid | |
| G204R (28883G>C) | nucleocapsid | |
| L84S (28144T>C) | ORF8 | |
| Y541C (17858A>G) | NSP13 (Helicase) | |
| P504L (17747C>T) | NSP13 (Helicase) | |
| S24L (27964C>T) | ORF8 | |

In some embodiments, the SARS-CoV-2 is variant B.1.1.7. In other embodiments, the SARS-CoV-2 is variant B.1.351. In other embodiments, the SARS-CoV-2 is variant P.1. In other embodiments, the SARS-CoV-2 is variant P.2. In other embodiments, the SARS-CoV-2 is variant B.1.207. In other embodiments, the SARS-CoV-2 is variant B.1.525. In other embodiments, the SARS-CoV-2 is variant Cluster 5.

### 5.4.2. Other Coronaviruses

In some embodiments, the coronavirus is a coronavirus other than SARS-CoV-2. For example, the coronavirus can be another coronavirus that can infect humans and/or animals, for example, SARS-CoV, MERS-CoV, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus, Human coronavirus OC43, Hedgehog coronavirus 1 (EriCoV), Beluga whale coronavirus SW1, Infectious bronchitis virus, Bulbul coronavirus HKU11, or Porcine coronavirus HKU15.

In some embodiments, the coronavirus is SARS-CoV.

In some embodiments, the coronavirus is MERS-CoV.

In some embodiments, the coronavirus is Human coronavirus 229E.

In some embodiments, the coronavirus is Human coronavirus NL63.

In some embodiments, the coronavirus is Miniopterus bat coronavirus 1.

In some embodiments, the coronavirus is Miniopterus bat coronavirus HKU8.

In some embodiments, the coronavirus is Porcine epidemic diarrhea virus.

In some embodiments, the coronavirus is Rhinolophus bat coronavirus HKU2.

In some embodiments, the coronavirus is Scotophilus bat coronavirus 512.

In some embodiments, the coronavirus is Betacoronavirus 1.

In some embodiments, the coronavirus is Human coronavirus HKU1.

In some embodiments, the coronavirus is Murine coronavirus.

In some embodiments, the coronavirus is Pipistrellus bat coronavirus HKU5.

In some embodiments, the coronavirus is Rousettus bat coronavirus HKU9.

In some embodiments, the coronavirus is Human coronavirus OC43.

In some embodiments, the coronavirus is Hedgehog coronavirus 1 (EriCoV).

In some embodiments, the coronavirus is Beluga whale coronavirus SW1.

In some embodiments, the coronavirus is Infectious bronchitis virus.

In some embodiments, the coronavirus is Bulbul coronavirus HKU11.

In some embodiments, the coronavirus is Porcine coronavirus HKU15.

In some embodiments, the coronavirus is Alphacoronavirus.

In some embodiments, the coronavirus is Colacovirus.

In some embodiments, the coronavirus is Bat coronavirus CDPHE15.

In some embodiments, the coronavirus is Decacovirus.

In some embodiments, the coronavirus is Bat coronavirus HKU10.

In some embodiments, the coronavirus is Rhinolophus ferrumequinum alphacoronavirus HuB-2013.

In some embodiments, the coronavirus is Duvinacovirus.

In some embodiments, the coronavirus is Luchacovirus.

In some embodiments, the coronavirus is Lucheng Rn rat coronavirus.

In some embodiments, the coronavirus is Minacovirus.

In some embodiments, the coronavirus is Ferret coronavirus.

In some embodiments, the coronavirus is Mink coronavirus 1.

In some embodiments, the coronavirus is Minunacovirus.

In some embodiments, the coronavirus is Myotacovirus.

In some embodiments, the coronavirus is Myotis ricketti alphacoronavirus Sax-2011.

In some embodiments, the coronavirus is Nyctacovirus.

In some embodiments, the coronavirus is Nyctalus velutinus alphacoronavirus SC-2013.

In some embodiments, the coronavirus is Pedacovirus.

In some embodiments, the coronavirus is Rhinacovirus.

In some embodiments, the coronavirus is Setracovirus.

In some embodiments, the coronavirus is NL63-related bat coronavirus strain BtKYNL63-9b.

In some embodiments, the coronavirus is Tegacovirus.

In some embodiments, the coronavirus is Alphacoronavirus 1 - type species.

In some embodiments, the coronavirus is Betacoronavirus.

In some embodiments, the coronavirus is Embecovirus.

In some embodiments, the coronavirus is China Rattus coronavirus HKU24.

In some embodiments, the coronavirus is Murine coronavirus - type species.

In some embodiments, the coronavirus is Hibecovirus.

In some embodiments, the coronavirus is Bat Hp-betacoronavirus Zhejiang2013.

In some embodiments, the coronavirus is Merbecovirus.

In some embodiments, the coronavirus is Middle East respiratory syndrome-related coronavirus (MERS-CoV).

In some embodiments, the coronavirus is Tylonycteris bat coronavirus HKU4.

In some embodiments, the coronavirus is Nobecovirus.

In some embodiments, the coronavirus is Rousettus bat coronavirus GCCDC1.

In some embodiments, the coronavirus is Sarbecovirus.

In some embodiments, the coronavirus is Severe acute respiratory syndrome-related coronavirus.

In some embodiments, the coronavirus is Severe acute respiratory syndrome coronavirus (SARS-CoV).

In some embodiments, the coronavirus is Deltacoronavirus.

In some embodiments, the coronavirus is Andecovirus.

In some embodiments, the coronavirus is Wigeon coronavirus HKU20.

In some embodiments, the coronavirus is Buldecovirus.

In some embodiments, the coronavirus is Bulbul coronavirus HKU11 -type species.

In some embodiments, the coronavirus is Munia coronavirus HKU13.

In some embodiments, the coronavirus is White-eye coronavirus HKU16.

In some embodiments, the coronavirus is Herdecovirus.

In some embodiments, the coronavirus is Night heron coronavirus HKU19.

In some embodiments, the coronavirus is Moordecovirus.

In some embodiments, the coronavirus is Common moorhen coronavirus HKU21.

In some embodiments, the coronavirus is Gammacoronavirus.

In some embodiments, the coronavirus is Cegacovirus.

In some embodiments, the coronavirus is Igacovirus.

In some embodiments, the coronavirus is Avian coronavirus-type species.

In some embodiments, the coronavirus is of another species under Orthocoronavirinae or Coronavirinae.

Additional exemplary coronaviruses are described, for example, in PCT publication No. WO 2016/012793.

### 5.5. Subject Populations

Subjects treatable with compounds and compositions described herein (e.g., vardenafil as monotherapy or in combination with an additional agent such as Remdesivir) includes but are not limited to mammals and birds. Exemplary mammals include human, swine, porcine, feline, bovine, canine, rabbit, ferret, bats, and/or any other mammals. The subject can also include other animals such as rodents.

In some embodiments, the subject is a human. For example, the subject can be a subject diagnosed with a coronavirus infection or suspected of having a coronavirus infection, e.g., a subject diagnosed with a SARS-CoV-2 infection or suspected of having a SARS-CoV-2 infection. Subjects can be adult or juvenile. In some embodiments, the subject is at least 12 years old.

Methods of the disclosure can include testing a subject for a coronavirus infection, for example before beginning treatment and/or after a period of treatment, for example to confirm that a subject has a coronavirus infection and/or to determine that a subject no longer has a coronavirus infection.

### 6. EXAMPLES

### 6.1. Example 1: Anti-Viral Activity of Vardenafil and Other Compounds

A study was performed to evaluate the antiviral effects of 14 drugs, including vardenafil, against SARS-CoV-2 as well as their toxicity. Antiviral potency was evaluated though titration in an MTT assay. The 14 drugs were initially selected from a computerized model designed to identify drugs having potential therapeutic activity against coronaviruses, in particular SARS-CoV-2.

### 6.1.1. Materials and Methods

### 6.1.1.1. Cell lines and Viruses

Lung fibroblast MRC5 cells (ATCC® CCL-171™) and were cultured in EMEM media supplemented with 10% FBS, 1% penicillin/streptomycin (Pen/strep). VeroE6 cells (Dunn School of Pathology, University of Oxford) were maintained in DMEM media supplemented with 10% FBS and 1% Pen/strep. The SARS-CoV-2 England strain (Dunn School of Pathology, University of Oxford) was propagated and titrated in VeroE6 cells.

### 6.1.1.2. In Vitro Antiviral Assay Setup

Cells were seeded at a density of 10,000 cells per well in a 96 well plate. After 24 hours (h), the cell medium was discarded and replaced with 100 µl of DMEM medium supplemented with 2% fetal calf serum in which was included 20 plaque forming units (PFU) of SARS-CoV-2/ per well. Study drugs were diluted in 2% FCS medium at 2x the desired final concentration in each well, so that the final working concentration in the well once added to the virus suspension was 1x.

Five initial drug concentrations were evaluated: 25 µM, 2.5 µM, 250 nM, 25 nM and 2.5 nM. The concentration of DMSO was adjusted across all dilutions for each drug to 0.05, 0.1 or 0.4% depending on the dilution factor applied. The controls included a cell only control and a relevant DMSO control (0.05%, 0.1% or 0.4% final concentration). For antiviral assays, a virus only control using 5, 20 and 100 PFU per well was included and for the MTT assay, a no cell control was included. 1 hour post infection, 100 µl of each antiviral dilution was added to 100 µl of virus suspension in each well and the plates were returned to incubator for 72 hours. For select compounds having greatest activity against SARS-CoV-2, a further study was carried out following this protocol, but with six concentrations using a fourfold dilution series. The concentrations evaluated were 25 µM, 6.25 µM, 1.56 µM, 0.39 nM, 0.097 nM and 0.024 nM.

IC₅₀ values were calculated by the Reed and Muench method.

### 6.1.1.3. Evaluation of Antiviral Activity by Development of Cytopathic Effects (CPE)

The potency of the drugs was assessed by examination of end points for development of CPE at 72 hours. The media of the cells was discarded and 200 µl of fixing and staining solution was added to each well. Following 1 hour incubation, the stain solution was removed. Each well was rinsed with fresh PBS and photographed.

### 6.1.1.4. Evaluation of Toxicity

Serially diluted compounds (⅒ dilution) were added in each well in the absence of virus and incubated for 72 hours, and at end point the CPE was recorded as above. In parallel, a duplicate plate was set up for an MTT assay (Abcam Ab 211091) carried out following the manufacturer’s recommendations. Briefly, the culture medium was discarded and 50 µl of fresh medium and MTT reagent was added to each well, including a background (no cell) control. The plates were returned to the 37° C. incubator for 3 hours before the MTT/media solution was discarded and 150 µl of MTT solvent was added to each well. The plates were incubated for a further 15 minutes in foil on a hula mixer and the absorbance recorded at 590 nm. The percentage of cytotoxicity was calculated as follows: (DMSO control - sample)/DMSO control, after replicate values were averaged and corrected for the background control (no cell control).

### 6.1.2. Results

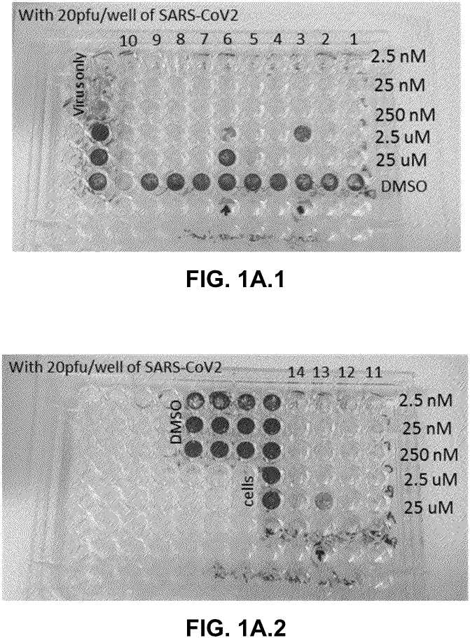

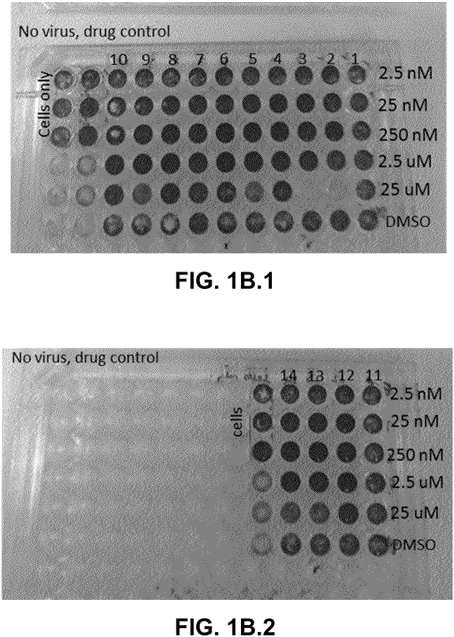

Results of the screening are shown in FIG. 1A.1-4C. A summary of antiviral potency and cytotoxicity of the study drugs is shown in Table 2.

TABLE 2

Antiviral Potency and Cytotoxicity

| | Drug | Initial titre^{a} | Repeat TCID₅₀ ^{b} | Minimum Cytotoxicity |
|---|---|---|---|---|
| 1 | Atazanavir | >25 µM | | >25 µM |
| 2 | Fludarabine phosphate | >25 µM | | 25 µM |
| 3 | Nelfinavir mesylate | 2.5 µM^{c} | 2.0 µM^{c} | 25 µM |
| 4 | Aztreonam | >25 µM | | >25 µM |
| 5 | Cobicistat | >25 µM | | >25 µM |
| 6 | Hydroxychloroquine | 25 µM | 2.9 µM | 25 µM |
| 7 | Tedizolid phosphate | >25 µM | | >25 µM |
| 8 | Indinavir sulfate | >25 µM | | >25 µM |
| 9 | Macitentan | >25 µM | | >25 µM |
| 10 | Nucleozin | >25 µM | | >25 µM |
| 11 | Lifitegrast | >25 µM | | >25 µM |
| 12 | Folinic acid calcium salt hydrate | >25 µM | | >25 µM |
| 13 | Vardenafil hydrochloride trihydrate | 25 µM | >25 µM | >25 µM |
| 14 | Rilpivirine hydrochloride | >25 µM | | >25 µM |

^{a}Minimum concentration of drug inhibiting development of CPE ^{b}TCID₅₀ measurement for drugs showing greatest initial efficacy on titration ^{c}Toxic at 25 µM

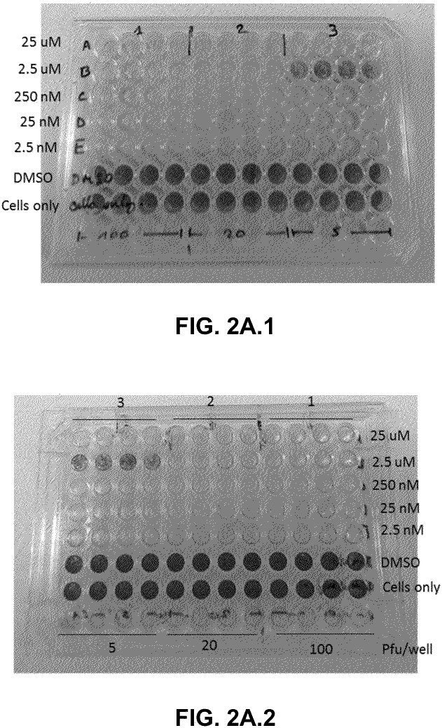

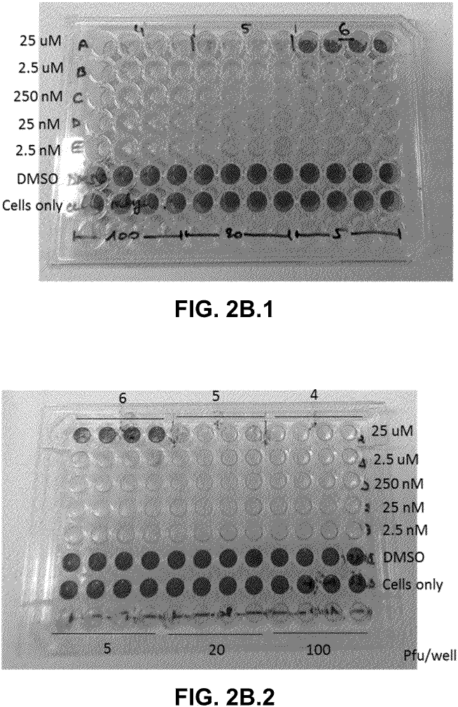

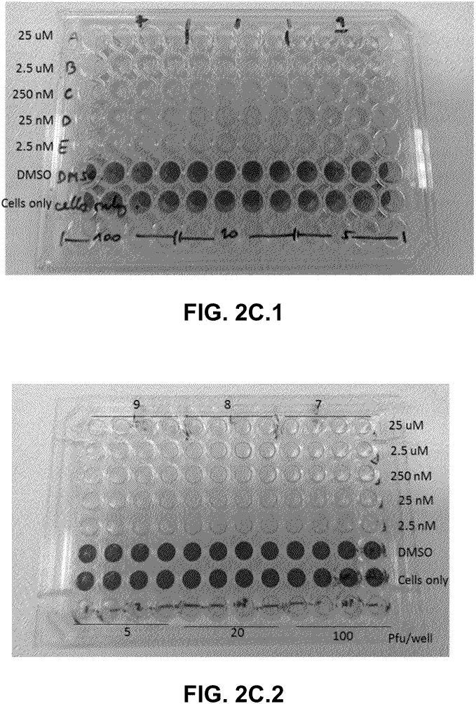

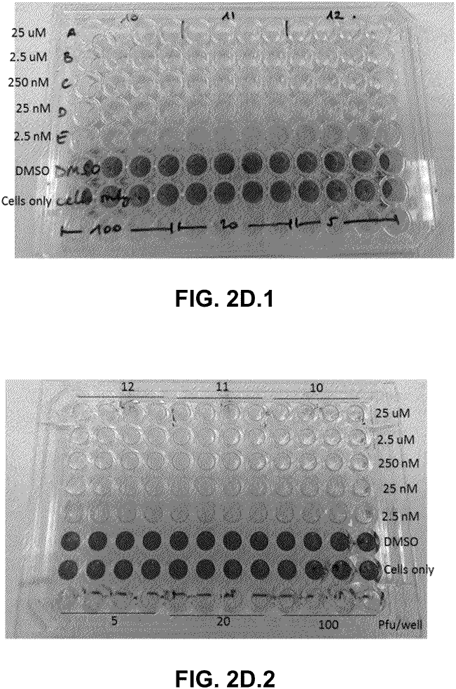

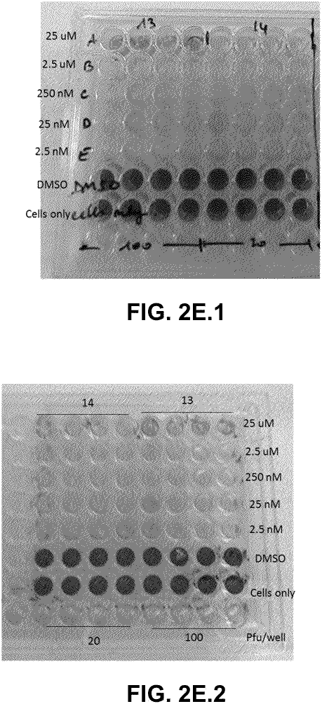

Initial screening of the compounds was carried out by titration of each antiviral in 5 serial ten-fold dilutions starting at 25 µMand challenge with 20 PFU SARS-CoV-2 per well. CPE in the Vero-E6 monolayer was scored in each well after a 72 hour incubation (see, FIG. 1A.1-1B.2). The study was repeated in 4 replicates of each compound dilution (see, FIG. 2A.1-2E.2). Consistently, compounds Nelfinavir mesylate, Hydroxychloroquine, and vardenafil hydrochloride trihydrate showed antiviral effects at high concentration (2.5 - 25 µM), while the other study drugs showed lower antiviral effects in this study.

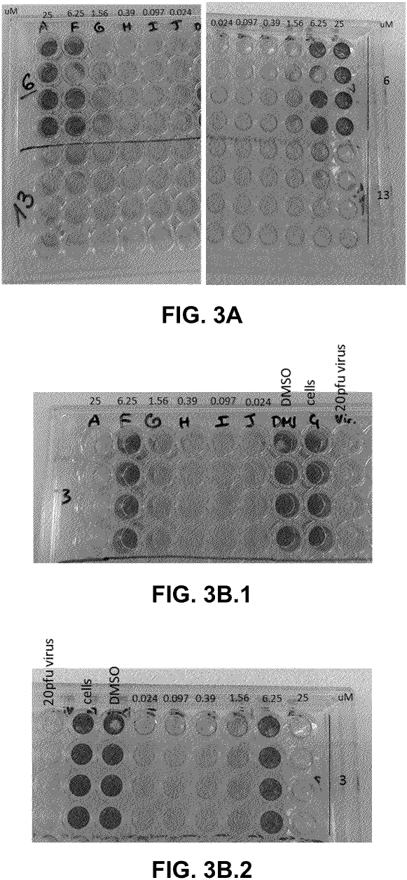

To more precisely estimate TCID₅₀ values of those three drugs showing high anti-viral activity in this assay, the drugs were re-assayed in narrower dilution steps in quadruplicate (see, FIG. 3A-3B.2). These yielded TCID₅₀ concentrations of 2.9 µM, 2.0 µM and >25 µMfor Nelfinavir mesylate, Hydroxychloroquine and vardenafil hydrochloride trihydrate, respectively.

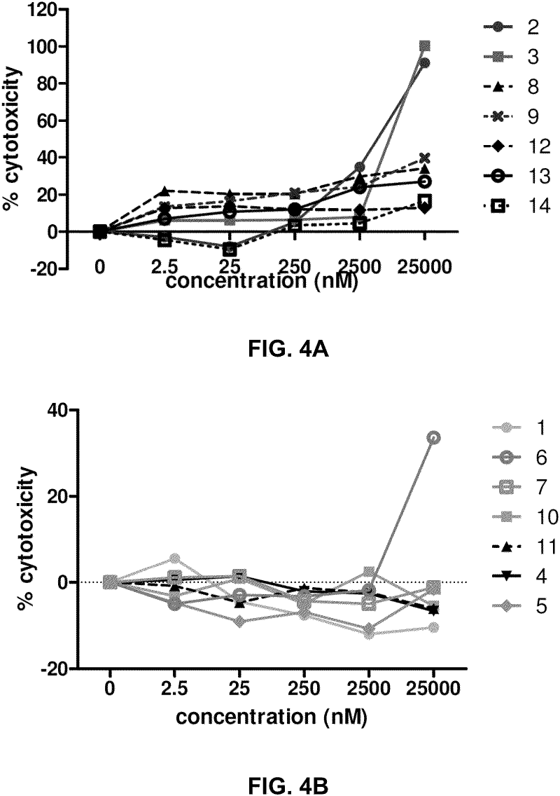

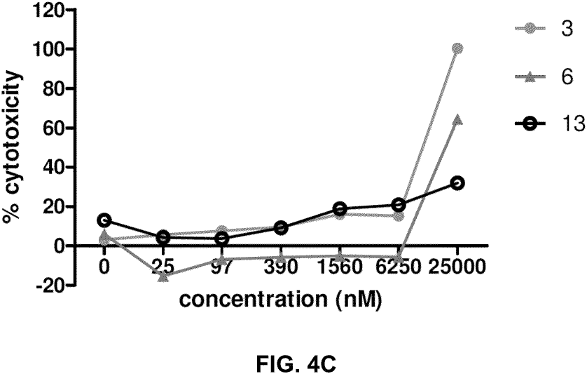

Fludarabine phosphate, nelfinavir mesylate and hydroxychloroquine showed cytotoxicity at a concentration of 25 µMin the MTT assay but none at 2.5 µM(see, FIGS. 4A-4C).

### 6.2. Example 2: Synergistic Activity of Vardenafil and Remdesivir

Following the initial screening of 14 drugs in Example 1, nelfinavir mesylate, hydroxychloroquine and vardenafil hydrochloride trihydrate were selected for further evaluation given that these three drugs showed the lowest IC₅₀ values out of the 14 tested drugs in Example 1. The antiviral activity of these drugs in combination with each other was evaluated on Vero-E6 cells. In addition, Remdesivir, previously documented to have antiviral activity against SARS-CoV-2, was also included in the study in combination with the three drugs.

### 6.2.1. Materials and Methods

### 6.2.1.1. Cell Lines and Viruses

Vero-E6 cells was maintained as in Example 1, and SARS-CoV-2 was propagated as in Example 1.

### 6.2.1.2. In Vitro Antiviral Assay Setup

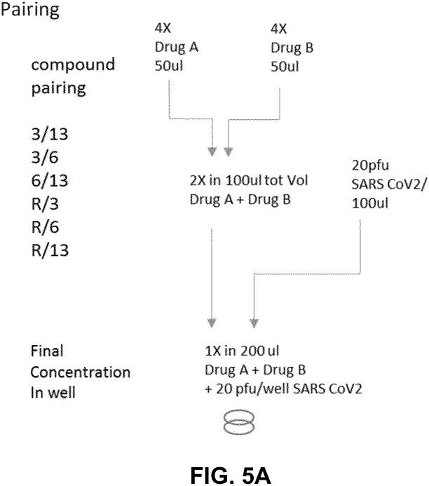

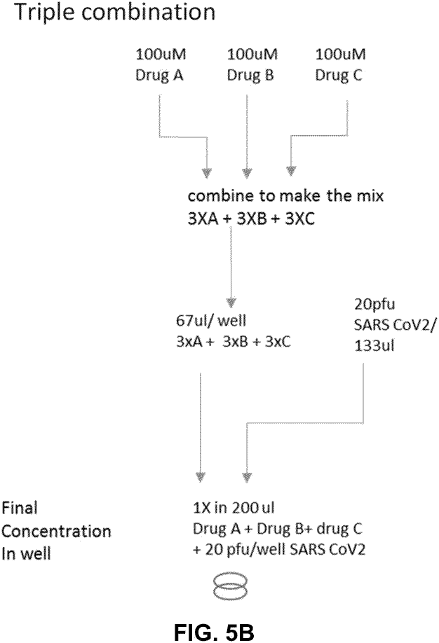

Cells were seeded at 10,000 cells per well in a 96 well plate. After 24 hours, the cell medium was discarded and replaced with 100 µl of 2% FCS medium in which was included 20 pfu of SARS-CoV-2/per well. Drugs were diluted in 2% FCS medium at 4x the desired final concentration in each well, so that the final working concentration in the well once added to the second drug and the virus suspension would be 1x (see, FIG. 5A). A fourfold dilution series was evaluated with final concentrations ranging from 25 µM, 6.25 µM, 1.56 µM, 0.39 nM, 0.097 nM and 0.024 nM in wells. The initial concentration selected for each drug when used in combination depended on its predetermined IC₅₀ value when used alone. Initial concentrations and IC₅₀ values for individual drugs when used alone are shown in Table 3A. Table 3B shows concentrations of Remdesivir used to determine the IC₅₀ value of Remdesivir alone. Concentrations of drugs used in pairwise combinations of this Example are shown in Table 3C and Table 3D.

The concentration of DMSO was adjusted across all dilutions for each drug to 0.1 %. The controls included a cell only and a relevant DMSO control at 0.1% final concentration. For antiviral assays, a virus only control using 20 pfu per well was additionally included. 1 hour post infection, 100 µl of antiviral dilution was added to 100 µl of virus suspension in each well and the plates were returned to incubator for 72 hours.

TABLE 3A

Preparation of Antiviral Drug Stock Dilutions for IC₅₀ Determination

| Drug | IC₅₀ | Stock concentration (mM) | Initial concentration in dilution (µM) | 4x concentration (µM) | Volume (µl) stock for 1 mM in 500 µl (T. Vol.) | Volume (µl) of 1 mM for 4x in 1200 µl (T. Vol.) |
|---|---|---|---|---|---|---|
| Nelfinavir mesylate | 390 nM | 51.97 | 1.56 | 6.25 | 9.62 | 75 |
| Hydroxychloroquine | 1579 nM | 84.85 | 6.25 | 25 | 5.89 | 300 |
| Vardenafil hydrochloride trihydrate | 6250 nM | 12.95 | 25 | 100 | 38.9 | 1200 |
| Remdesivir | 1.56 µM | 10 | 6.25 | 25 | 5 | 300 |

TABLE 3B

Concentrations of vardenafil (compound 13), nelfinavir (compound 3), and hydroxychloroquine (compound 6) in pairwise combinations

| | vardenafil (µM) | hydroxychloroquine (µM) | nelfinavir (µM) |
|---|---|---|---|
| 1 | 25 | 6.25 | 1.56 |
| 2 | 6.25 | 1.56 | 0.39 |
| 3 | 1.56 | 0.39 | 0.097 |
| 4 | 0.39 | 0.097 | 0.024 |
| 5 | 0.097 | 0.024 | 0.006 |

TABLE 3C

Concentrations of Remdesivir used to determine Remdesivir IC50 (alone)

| | Remdesivir (µM) |
|---|---|
| 1 | 25 |
| 2 | 6.25 |
| 3 | 1.56 |
| 4 | 0.39 |
| 5 | 0.097 |
| 6 | 0.024 |

TABLE 3D

Concentrations of Remdesivir, vardenafil (compound 13), nelfinavir (compound 3), and hydroxychloroquine (compound 6) in pairwise combinations

| | Remdesivir (µM) | vardenafil (µM) | hydroxychloroquine (µM) | nelfinavir (µM) |
|---|---|---|---|---|
| 1 | 6.25 | 25 | 6.25 | 1.56 |
| 2 | 1.56 | 6.25 | 1.56 | 0.39 |
| 3 | 0.39 | 1.56 | 0.39 | 0.097 |
| 4 | 0.097 | 0.39 | 0.097 | 0.024 |
| 5 | 0.024 | 0.097 | 0.024 | 0.006 |
| 6 | 0.006 | 0.024 | 0.006 | 0.0015 |

To combine all three drugs, they were first mixed to 3x the highest concentration needed then diluted fourfold. 67 µl of 3x mix of drugs was added to each well and 20 pfu/ well of SARS-CoV-2 was prepared in 133 µl/well, so that the final concentration of drugs was 1x for each drug and 20 pfu/well of virus (see, FIG. 5B). Table 3E lists the concentrations of drugs used to evaluate the three drugs in combination.

TABLE 3E

Concentrations of vardenafil (compound 13), nelfinavir (compound 3), and hydroxychloroquine (compound 6) in triple combination

| | vardenafil (µM) | hydroxychloroquine (µM) | nelfinavir (µM) |
|---|---|---|---|
| 1 | 25 | 6.25 | 1.56 |
| 2 | 6.25 | 1.56 | 0.39 |
| 3 | 1.56 | 0.39 | 0.097 |
| 4 | 0.39 | 0.097 | 0.024 |
| 5 | 0.097 | 0.024 | 0.006 |
| 6 | 0.024 | 0.006 | 0.0015 |

### 6.2.1.3. Evaluation of Antiviral Activity by Development of Cytopathic Effects (CPE)

The efficacy of the antivirals was assessed by the capacity of treated cells to resist the viral challenge over 72 hours and determined at the end point by observation of cytopathic effect. The media of the cells was discarded and 200 µl of fixing and staining solution was added to each well. Following a 30 minute incubation, the stain solution was removed, each well rinsed with fresh PBS and photographed. The IC₅₀ of each compound was assessed using Graphpad Prism.

IC₅₀ values calculated for each combination were compared with IC₅₀ values for each antiviral drug evaluated individually. Synergy was calculated as the ratio of IC₅₀ values of the drugs evaluated in combination with half of the IC₅₀ values of the drugs evaluated individually, assuming a simple additive effect on antiviral activity, e.g.,

$$\text{Pairwise} \text{Synergism=0} \text{.5(Drug} \text{1} {\text{IC}}_{50 i n d i v .} + D r u g 2 I {C}_{50 i n d i v .} ) /$$

$$\left(\text{Drug 1} {\text{IC}}_{50 \text{comb} \text{.}} + \text{Drug} \text{2} {\text{IC}}_{50 \text{comb} \text{.}}\right)$$

### 6.2.2. Results

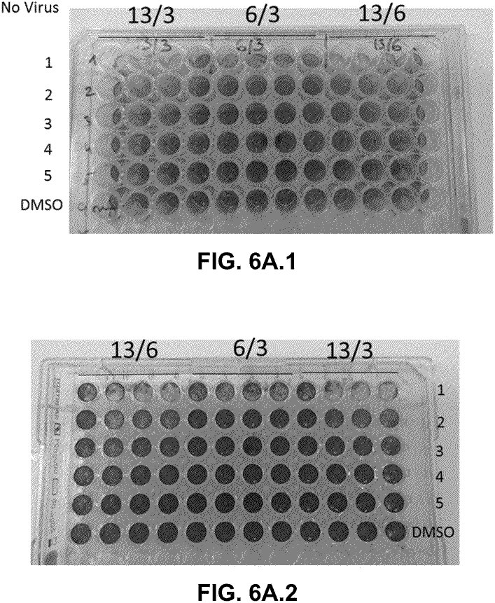

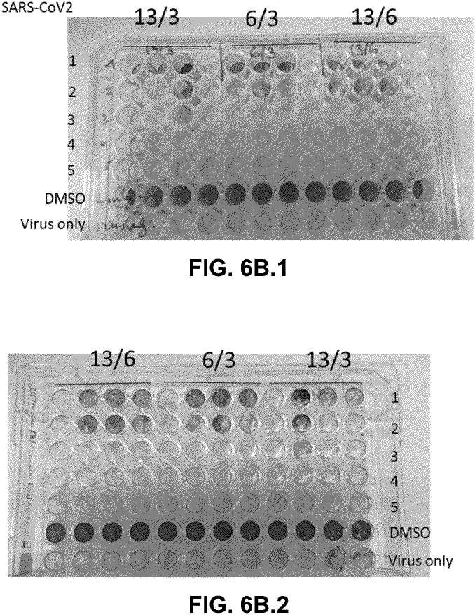

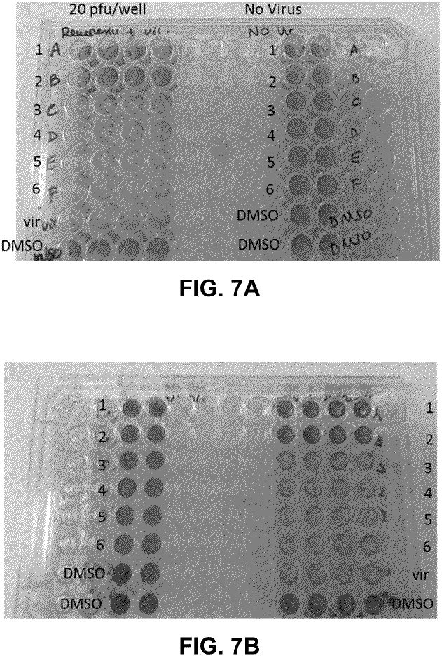

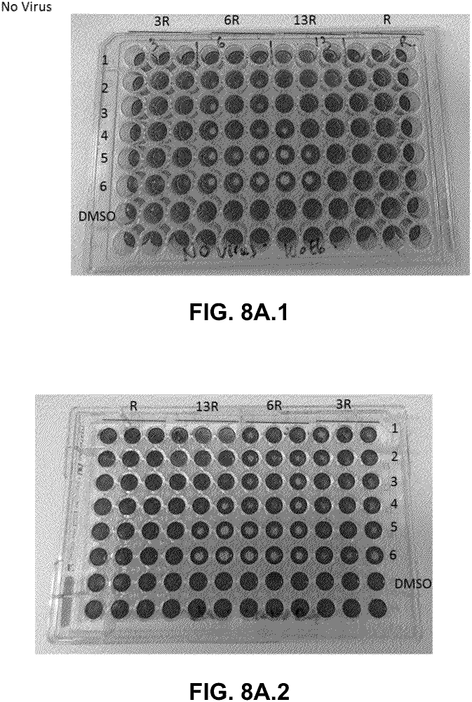

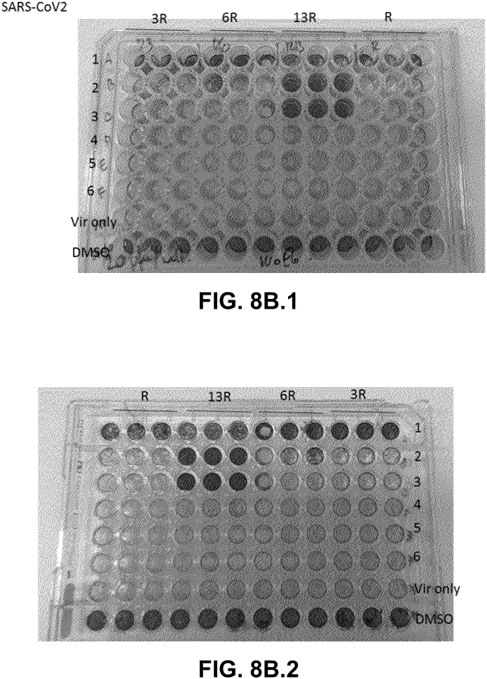

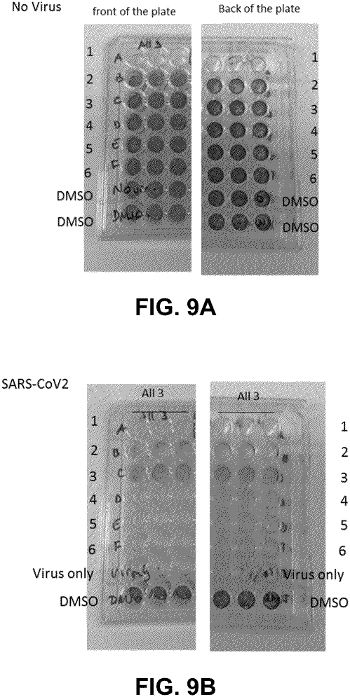

Assay plates for pairwise combinations of vardenafil (compound 13), nelfinavir (compound 3) and hydroxychloroquine (compound 6) are shown in FIG. 6A.1-6B.2 (i.e., combinations 3+13, 3+6, 6+13). Drug concentrations used in the plates shown in FIG. 6A.1-6B.2 are set forth in Table 3B. Assay plate for Remdesivir alone is shown in FIGS. 7A-7B. Drug concentrations used in the plate shown in FIGS. 7A-7B are set forth in Table 3C. Assay plates for pairwise combinations of Remdesivir (R) with vardenafil (compound 13), nelfinavir (compound 3), and hydroxychloroquine (compound 6) are shown in FIG. 8A.1-8B.2 (i.e., combinations 3+R, 6+R, 13+R and R only). Drug concentrations used in the plates shown in FIG. 8A.1-8B.2 are shown in Table 3D. Assay plates for the triple combinations of vardenafil (compound 13), nelfinavir (compound 3) and hydroxychloroquine (compound 6) are shown in FIGS. 9A-9B (i.e., combination 3+6+13). Drug concentrations used in the plates shown in FIGS. 9A-9B are set forth in Table 3E.

IC₅₀ values calculated from the plates for combinations of study drugs are shown in Table 4 and Table 5.

TABLE 4

IC50 Values (µM)for Antiviral Drugs in Pairwise Combination

| | | IC₅₀ measured when each drug used individually | | | IC₅₀ measured when drugs used in combination | | |
|---|---|---|---|---|---|---|---|
| Drug 1 | Drug 2 | Drug 1 IC₅₀ (µM) | Drug 2 IC₅₀ (µM) | Drug 1 IC₅₀: Drug 2 IC₅₀ | Drug 1 IC₅₀ (µM) | Drug 2 IC₅₀ (µM) | Synergism |
| Vardenafil hydrochloride trihydrate | Nelfinavir mesylate | 6.25 | 0.39 | 16:1 | 1.66 | 0.1033 | 1.88 |
| Vardenafil hydrochloride trihydrate | Hydroxychloroquine | 6.25 | 1.579 | 4:1 | 1.56 | 0.39 | 2.00 |
| Hydroxychloroquine | Nelfinavir mesylate | 1.579 | 0.39 | 4:1 | 0.999 | 0.2035 | 0.79 |
| Vardenafil hydrochloride trihydrate | Remdesivir | 6.25 | 1.56 | 4:1 | 0.4149 | 0.1033 | 7.53 |

TABLE 4

IC50 Values (µM)for Antiviral Drugs in Pairwise Combination

| | | IC₅₀ measured when each drug used individually | | | IC₅₀ measured when drugs used in combination | | |
|---|---|---|---|---|---|---|---|
| Drug 1 | Drug 2 | Drug 1 IC₅₀ (µM) | Drug 2 IC₅₀ (µM) | Drug 1 IC₅₀: Drug 2 IC₅₀ | Drug 1 IC₅₀ (µM) | Drug 2 IC₅₀ (µM) | Synergism |
| Hydroxychloroquine | Remdesivir | 1.579 | 1.56 | 1:1 | 0.78 | 0.78 | 1.01 |
| Nelfinavir mesylate | Remdesivir | 0.39 | 1.56 | 1:4 | 0.39 | 1.66 | 0.50 |

TABLE 5

IC50 Values (µM)for Antiviral Drugs in Triple Combination

| | | | IC₅₀ measured when drugs used in combination | | | |
|---|---|---|---|---|---|---|
| Drug 1 | Drug 2 | Drug 3 | Drug 1 IC₅₀ | Drug 2 IC₅₀ | Drug 3 IC₅₀ | Synergism |
| vardenafil hydrochloride trihydrate | Hydroxychloroquine | Nelfinavir mesylate | 15.76 | 3.922 | 0.9779 | 0.13 |

Combinations of both Nelfinavir mesylate and Hydroxychloroquine with vardenafil hydrochloride trihydrate showed about 2-fold greater potency than when assayed individually, while the combination of Nelfinavir mesylate and Hydroxychloroquine showed an approximately additive effect.

The combination of Remdesivir with vardenafil hydrochloride trihydrate showed a surprisingly strongly synergistic effect (7.53x synergistic effect), with IC₅₀ values approximately a log lower than calculated from an additive effect. The other Remdesivir combinations did not show as strong of a synergistic effect in this study (1x or less synergistic effect), while the triple combination of Nelfinavir mesylate, Hydroxychloroquine, and vardenafil hydrochloride trihydrate showed a lack of synergy (0.13x synergistic effect)..

This Example shows that combinations of study drugs can show a degree of synergistic activity, particularly vardenafil and Remdesivir, where IC₅₀ values were surprisingly well into the nanomolar range when combined (415 nM and 103 nM), over ten-fold lower than when used individually. Without being bound by theory, it is believed that the combination of vardenafil and Remdesivir for treating a coronavirus infection can be advantageous over Remdesivir alone because, for example, the combination may allow for a smaller dose of Remdesivir to be used while achieving a therapeutic benefit. Remdesivir has known serious side effects, including elevated liver transaminase, which can be indicative of liver inflammation and/or damage (see, Veklury® prescribing information). Combination therapies having a synergistic effect compared to their component therapeutic agents can allow a lower dose of the therapeutic agents to be used, with the potential for reduced side effects and/or increased efficacy compared to use of single therapeutic agents alone. Again without being bound by theory, it is believed that a dose of vardenafil which is therapeutically effective as monotherapy can be combined with a dose of Remdesivir which is therapeutically effective as monotherapy to provide a combined therapy having increased efficacy (e.g., a synergistic effect).

### 7. SPECIFIC EMBODIMENTS

### 7.1. Specific Embodiments: Group 1

Various aspects of the present disclosure are described in the embodiments set forth in the following numbered paragraphs, where reference to a previous numbered embodiment refers to a previous numbered embodiment in this Section 7.1.
1. A compound for treating diseases and/or conditions caused by, arising from, and/or associated with coronavirus in a subject in need thereof.
2. The compound of embodiment 1, wherein the compound targets one or more proteins of the coronavirus comprising 3CL-protease and/or RNA-dependent RNA polymerase (RdRP, RDR, or called RNA replicase).
3. The compound of embodiment 1, wherein the compound targets coronavirus in general without any specific protein(s) thereof.
4. The compound of embodiment 1, wherein the compound is one or more selected from lopinavir, ritonavir, atazanavir, indinavir sulfate, nelfinavir mesylate, ceftaroline fosamil, leucal (or called leucovorin), aztreonam, cangrelor, vardenafil, fludarabine, eltrombopag, tedizolid, macitentan, cobicistat, and/or lifitegrast, and any derivatives thereof.
5. The compound of embodiment 1, wherein the compound is formulated into one or more of the following forms: solution, tablet, capsule, granules, powders, crystals, gel, liquid, solvent, paste, pellet, or any other suitable forms.
6. The compound of embodiment 5, wherein the compound is administered to said subject in need thereof via oral, intraperitoneal, intramuscular, intravenous, inhalation, topical, buccal, injection, nasal, transdermal, intradermal, translingual, urethral, vaginal, enteral, rectal, ophthalmic, subcutaneous, irrigation, mouth, throat, transmucosal, intravitreal, otic, perfusion, perivascular, and/or sublingual, or any other suitable routes of administration.
7. The compound of embodiment 1, wherein said diseases and/or conditions that the present compound or composition is capable of treating comprise one or more of severe acute respiratory syndrome (SARS), Middle East respiratory syndrome (MERS), Coronavirus disease 2019 (COVID-19), pulmonary infection, respiratory tract infections, cold, fever, upper and/or lower respiratory tract infections and/or diseases, pneumonia, diarrhea, dry cough, dyspnea, sore throat, any other infections.
8. The compound of embodiment 1, wherein said coronavirus comprises one or more of SARS-CoV, MERS-CoV, Covid-19, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus, Human coronavirus OC43, Hedgehog coronavirus 1 (EriCoV), Beluga whale coronavirus SW1, Infectious bronchitis virus, Bulbul coronavirus HKU11, Porcine coronavirus HKU15, and/or the following list:
   Alphacoronavirus
      Colacovirus
      Bat coronavirus CDPHE15
   Decacovirus
      Bat coronavirus HKU10
      Rhinolophus ferrumequinum alphacoronavirus HuB-2013
   Duvinacovirus
      Human coronavirus 229E
   Luchacovirus
      Lucheng Rn rat coronavirus
   Minacovirus
      Ferret coronavirus
      Mink coronavirus 1
   Minunacovirus
      Miniopterus bat coronavirus 1
      Miniopterus bat coronavirus HKU8
   Myotacovirus
      Myotis ricketti alphacoronavirus Sax-2011
   Nyctacovirus
      Nyctalus velutinus alphacoronavirus SC-2013
   Pedacovirus
      Porcine epidemic diarrhea virus
      Scotophilus bat coronavirus 512
   Rhinacovirus
      Rhinolophus bat coronavirus HKU2
   Setracovirus
      Human coronavirus NL63
      NL63-related bat coronavirus strain BtKYNL63-9b
   Tegacovirus
      Alphacoronavirus 1 – type species
   Betacoronavirus
      Embecovirus
      Betacoronavirus 1
      Human coronavirus OC43
      China Rattus coronavirus HKU24
      Human coronavirus HKU1
      Murine coronavirus - type species
   Hibecovirus
      Bat Hp-betacoronavirus Zhejiang2013
   Merbecovirus
      Hedgehog coronavirus 1
      Middle East respiratory syndrome-related coronavirus (MERS-CoV)
      Pipistrellus bat coronavirus HKU5
      Tylonycteris bat coronavirus HKU4
   Nobecovirus
      Rousettus bat coronavirus GCCDC1
      Rousettus bat coronavirus HKU9
   Sarbecovirus
      Severe acute respiratory syndrome-related coronavirus
      Severe acute respiratory syndrome coronavirus (SARS-CoV)
      Severe acute respiratory syndrome coronavirus 2 (SARS-CoV-2, Wuhan 2020 nCOV)
   Deltacoronavirus
      Andecovirus
      Wigeon coronavirus HKU20
   Buldecovirus
      Bulbul coronavirus HKU11 – type species
      Porcine coronavirus HKU15
      Munia coronavirus HKU13
      White-eye coronavirus HKU16
   Herdecovirus
      Night heron coronavirus HKU19
   Moordecovirus
      Common moorhen coronavirus HKU21
   Gammacoronavirus
      Cegacovirus
      Beluga whale coronavirus SW1
      Igacovirus
      Avian coronavirus-type species,
   and/or any other species under Orthocoronavirinae or Coronavirinae.
9. The compound of embodiment 1, wherein said subject comprises mammals, birds and other animals, and wherein said mammals comprise human, swine, porcine, feline, bovine, canine, rabbit, ferret, bats, and/or any other mammals, and wherein said other animals comprise rodents.
10. A composition comprising the compound of any of the preceding embodiments for treating diseases and/or conditions caused by, arising from, and/or associated with coronavirus in a subject.
11. The composition of embodiment 10, further comprising one or more of pharmaceutically acceptable carrier, ester, salt, acid, alkali, vehicle, excipient, binder, agent, diluent, stabilizer, emulsifier, absorbent, surfactant, lubricant, disintegrant, glidant, buffer, filler, gels, preservative, dissolution, liquid, solvent, medium, and/or any other suitable materials / complex.
12. The composition of embodiment 10, wherein said diseases and/or conditions that the present compound or composition is capable of treating comprise one or more of severe acute respiratory syndrome (SARS), Middle East respiratory syndrome (MERS), Coronavirus disease 2019 (COVID-19), pulmonary infection, respiratory tract infections, cold, fever, upper and/or lower respiratory tract infections and/or diseases, pneumonia, diarrhea, dry cough, dyspnea, sore throat, any other infections.
13. The composition of embodiment 10, wherein said coronavirus comprises one or more of SARS-CoV, MERS-CoV, Covid-19, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus, Human coronavirus OC43, Hedgehog coronavirus 1 (EriCoV), Beluga whale coronavirus SW1, Infectious bronchitis virus, Bulbul coronavirus HKU11, Porcine coronavirus HKU15, and/or the following list:
   Alphacoronavirus
      Colacovirus
      Bat coronavirus CDPHE15
   Decacovirus
      Bat coronavirus HKU10
      Rhinolophus ferrumequinum alphacoronavirus HuB-2013
   Duvinacovirus
      Human coronavirus 229E
   Luchacovirus
      Lucheng Rn rat coronavirus
   Minacovirus
      Ferret coronavirus
      Mink coronavirus 1
   Minunacovirus
      iniopterus bat coronavirus 1
      Miniopterus bat coronavirus HKU8
   Myotacovirus
      Myotis ricketti alphacoronavirus Sax-2011
   Nyctacovirus
      Nyctalus velutinus alphacoronavirus SC-2013
   Pedacovirus
      Porcine epidemic diarrhea virus
      Scotophilus bat coronavirus 512
   Rhinacovirus
      Rhinolophus bat coronavirus HKU2
   Setracovirus
      Human coronavirus NL63
      NL63-related bat coronavirus strain BtKYNL63-9b
   Tegacovirus
      Alphacoronavirus 1 - type species
   Betacoronavirus
      Embecovirus
      Betacoronavirus 1
      Human coronavirus OC43
      China Rattus coronavirus HKU24
      Human coronavirus HKU1
      Murine coronavirus - type species
   Hibecovirus
      Bat Hp-betacoronavirus Zhejiang2013
   Merbecovirus
      -Hedgehog coronavirus 1
      Middle East respiratory syndrome-related coronavirus (MERS-CoV)
      Pipistrellus bat coronavirus HKU5
      Tylonycteris bat coronavirus HKU4
   Nobecovirus
      Rousettus bat coronavirus GCCDC1
      Rousettus bat coronavirus HKU9
   Sarbecovirus
      Severe acute respiratory syndrome-related coronavirus
      Severe acute respiratory syndrome coronavirus (SARS-CoV)
      Severe acute respiratory syndrome coronavirus 2 (SARS-CoV-2, Wuhan 2020 nCOV)
   Deltacoronavirus
      Andecovirus
      Wigeon coronavirus HKU20
   Buldecovirus
      ulbul coronavirus HKU11 – type species
      Porcine coronavirus HKU15
      Munia coronavirus HKU13
      White-eye coronavirus HKU16
   Herdecovirus
      Night heron coronavirus HKU19
   Moordecovirus
      Common moorhen coronavirus HKU21
   Gammacoronavirus
      Cegacovirus
      Beluga whale coronavirus SW1
      Igacovirus
      Avian coronavirus-type species,
   and/or any other species under Orthocoronavirinae or Coronavirinae.
14. The composition of embodiment 10, wherein said subject comprises mammals, birds and other animals, and wherein said mammals comprise human, swine, porcine, feline, bovine, canine, rabbit, ferret, bats, and/or any other mammals, and wherein said other animals comprise rodents.
15. A method of treating a coronavirus infection, diseases and/or conditions caused by, arising from, and/or associated with coronaviruses comprising administering the compound or the composition comprising the compound of any of the preceding embodiments to a subject in need thereof.
16. The method of embodiment 15, wherein the compound or composition targets one or more proteins of the coronavirus comprising 3CL-protease, and/or RNA-dependent RNA polymerase (RdRP, RDR, or called RNA replicase).
17. The method of embodiment 15, wherein the compound or composition targets said coronavirus in general without any specific protein(s) thereof.
18. The method of embodiment 15, wherein the compound is one or more of: lopinavir, ritonavir, atazanavir, indinavir sulfate, nelfinavir mesylate, ceftaroline fosamil, leucal (or called leucovorin), aztreonam, cangrelor, vardenafil, fludarabine, eltrombopag, tedizolid, macitentan, cobicistat, and/or lifitegrast, and any derivatives thereof.
19. The method of embodiment 15, wherein the compound is formulated into one or more of the following forms: solution, tablet, capsule, granules, powders, crystals, gel, liquid, solvent, paste, pellet, or any other suitable forms.
20. The method of embodiment 15, wherein the compound or composition is administered to said subject in need thereof via oral, intraperitoneal, intramuscular, intravenous, inhalation, topical, buccal, injection, nasal, transdermal, intradermal, translingual, urethral, vaginal, enteral, rectal, ophthalmic, subcutaneous, irrigation, mouth, throat, transmucosal, intravitreal, otic, perfusion, perivascular, and/or sublingual, or any other suitable routes of administration.
21. The method of embodiment 15, wherein said composition comprises one or more of pharmaceutically acceptable carrier, ester, salt, acid, alkali, vehicle, excipient, binder, agent, diluent, stabilizer, emulsifier, absorbent, surfactant, lubricant, disintegrant, glidant, buffer, filler, gels, preservative, dissolution, liquid, solvent, medium, and/or any other suitable materials / complex.
22. The method of embodiment 15, wherein said diseases and/or conditions comprise one or more of severe acute respiratory syndrome (SARS), Middle East respiratory syndrome (MERS), Coronavirus disease 2019 (COVID-19), pulmonary infection, respiratory tract infections, cold, fever, upper and/or lower respiratory tract infections and/or diseases, pneumonia, diarrhea, dry cough, dyspnea, sore throat, any other infections.
23. The method of embodiment 15, wherein said coronavirus comprises one or more of SARS-CoV, MERS-CoV, Covid-19, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus, Human coronavirus OC43, Hedgehog coronavirus 1 (EriCoV), Beluga whale coronavirus SW1, Infectious bronchitis virus, Bulbul coronavirus HKU11, Porcine coronavirus HKU15, and/or the following list:
   Alphacoronavirus
      Colacovirus
      Bat coronavirus CDPHE15
   Decacovirus
      Bat coronavirus HKU10
      Rhinolophus ferrumequinum alphacoronavirus HuB-2013
   Duvinacovirus
      Human coronavirus 229E
   Luchacovirus
      Lucheng Rn rat coronavirus
   Minacovirus
      Ferret coronavirus
      Mink coronavirus 1
   Minunacovirus
      Miniopterus bat coronavirus 1
      Miniopterus bat coronavirus HKU8
   Myotacovirus
      Myotis ricketti alphacoronavirus Sax-2011
   Nyctacovirus
      Nyctalus velutinus alphacoronavirus SC-2013
   Pedacovirus
      Porcine epidemic diarrhea virus
      Scotophilus bat coronavirus 512
   Rhinacovirus
      Rhinolophus bat coronavirus HKU2
   Setracovirus
      Human coronavirus NL63
      NL63-related bat coronavirus strain BtKYNL63-9b
   Tegacovirus:
      Alphacoronavirus 1 - type species
   Betacoronavirus
      Embecovirus
      Betacoronavirus 1
      Human coronavirus OC43
      China Rattus coronavirus HKU24
      Human coronavirus HKU1
      Murine coronavirus - type species
   Hibecovirus
      Bat Hp-betacoronavirus Zhejiang2013
   Merbecovirus
      Hedgehog coronavirus 1
      Middle East respiratory syndrome-related coronavirus (MERS-CoV)
      Pipistrellus bat coronavirus HKU5
      Tylonycteris bat coronavirus HKU4
   Nobecovirus
      Rousettus bat coronavirus GCCDC1
      Rousettus bat coronavirus HKU9
   Sarbecovirus
      Severe acute respiratory syndrome-related coronavirus
      Severe acute respiratory syndrome coronavirus (SARS-CoV)
      Severe acute respiratory syndrome coronavirus 2 (SARS-CoV-2, Wuhan 2020 nCOV)
   Deltacoronavirus
      Andecovirus
      Wigeon coronavirus HKU20
   Buldecovirus
      Bulbul coronavirus HKU11 - type species
      Porcine coronavirus HKU15
      Munia coronavirus HKU13
      White-eye coronavirus HKU16
   Herdecovirus
      Night heron coronavirus HKU19
   Moordecovirus
      Common moorhen coronavirus HKU21
   Gammacoronavirus
      Cegacovirus
      Beluga whale coronavirus SW1
      Igacovirus
      Avian coronavirus-type species,
   and/or any other species under Orthocoronavirinae or Coronavirinae.
24. The method of embodiment 15, wherein said subject comprises mammals, birds and other animals, and wherein said mammals comprise human, swine, porcine, feline, bovine, canine, rabbit, ferret, bats, and/or any other mammals, and wherein said other animals comprise rodents.

### 7.2. Specific Embodiments: Group 2

Further aspects of the present disclosure are described in the embodiments set forth in the following numbered paragraphs, where reference to a previous numbered embodiment refers to a previous numbered embodiment in this Section 7.2.
1. Use of vardenafil for the manufacture of a medicament for treating a coronavirus infection in a subject.
2. The use according to embodiment 1, wherein the medicament is for administration as monotherapy.
3. The use according to embodiment 1, wherein the medicament is for administration in combination with one or more additional agents.
4. The use according to embodiment 3, wherein the medicament comprises the one or more additional agents.
5. The use according to embodiment 3, wherein the medicament does not comprise the one or more additional agents.
6. Use of vardenafil and one or more additional agents for the manufacture of a medicament comprising both vardenafil and the one or more additional agents for treating a coronavirus infection in a subject.
7. The use according to any one of embodiments 3 to 6, wherein the one or more additional agents comprise an antiviral agent.
8. The use according to embodiment 7, wherein the antiviral agent targets RNA-dependent RNA polymerase (RdRP).
9. The use according to embodiment 7 or embodiment 8, wherein the antiviral agent is Remdesivir.
10. The use according to embodiment 7, wherein the antiviral agent is a protease inhibitor.
11. The use according to embodiment 10, wherein the antiviral agent is amprenavir, atazanavir, indinavir, darunavir, fosamprenavir, nelfinavir, ritonavir, saquinavir, tipranavir or lopinavir.
12. The use according to embodiment 7, wherein the antiviral agent is a nucleoside analog.
13. The use according to embodiment 12, wherein the antiviral agent is Remdesivir, ribavirin, didanosine, vidarabine, galidesivir, , emtricitabine, lamivudine, zalcitabine, abacavir, acyclovir, entecavir, stavudine, telbivudine, zidovudine, idoxuridine, or trifluridine.
14. The use according to embodiment 7, wherein the antiviral agent is an interferon.
15. The use according to embodiment 14, wherein the antiviral agent is interferon beta 1b.
16. Use of an antiviral agent for the manufacture of a medicament for treating a coronavirus infection in a subject, wherein the medicament is for administration in combination with vardenafil.
17. The use according to embodiment 16, wherein the medicament comprises the vardenafil.
18. The use according to embodiment 16, wherein the medicament does not comprise the vardenafil.
19. The use according to any one of embodiments 16 to 18, wherein the antiviral agent targets RNA-dependent RNA polymerase (RdRP).
20. The use according to any one of embodiments 16 to 19, wherein the antiviral agent is Remdesivir.
21. The use according to any one of embodiments 16 to 18, wherein the antiviral agent is a protease inhibitor.
22. The use according to embodiment 21, wherein the antiviral agent is amprenavir, atazanavir, indinavir, darunavir, fosamprenavir, nelfinavir, ritonavir, saquinavir, tipranavir or lopinavir.
23. The use according to any one of embodiments 16 to 18, wherein the antiviral agent is a nucleoside analog.
24. The use according to embodiment 23, wherein the antiviral agent is Remdesivir, ribavirin, didanosine, vidarabine, galidesivir, emtricitabine, lamivudine, zalcitabine, abacavir, acyclovir, entecavir, stavudine, telbivudine, zidovudine, idoxuridine, or trifluridine.
25. The use according to any one of embodiments 16 to 18, wherein the antiviral agent is an interferon.
26. The use according to embodiment 25, wherein the antiviral agent is interferon beta 1b.
27. The use according to any one of embodiments 1 to 26, wherein the medicament is a solution, tablet, capsule, granule, powder, crystal, gel, liquid, suspension, paste, or pellet.
28. Vardenafil for use in the treatment of a coronavirus infection in a subject.
29. The vardenafil for use according to embodiment 28, wherein the vardenafil is to be administered as monotherapy for treatment of the coronavirus infection.
30. The vardenafil for use according to embodiment 28, wherein the vardenafil is to be administered in combination with one or more additional agents for treatment of the coronavirus infection.
31. The vardenafil for use according to embodiment 30, wherein the vardenafil and the one or more additional agents are in a single pharmaceutical composition.
32. The vardenafil for use according to embodiment 31, wherein the pharmaceutical composition is a solution, tablet, capsule, granule, powder, crystal, gel, liquid, suspension, paste, or pellet.
33. The vardenafil for use according to embodiment 30, wherein the vardenafil and the one or more additional agents are in different pharmaceutical compositions.
34. The vardenafil for use according to embodiment 33, wherein the pharmaceutical compositions are each independently a solution, tablet, capsule, granule, powder, crystal, gel, liquid, suspension, paste, or pellet.
35. The vardenafil for use according to any one of embodiments 30 to 34, wherein the one or more additional agents comprise an antiviral agent.
36. The vardenafil for use according to embodiment 35, wherein the antiviral agent targets RNA-dependent RNA polymerase (RdRP).
37. The vardenafil for use according to embodiment 35 or embodiment 36, wherein the antiviral agent is Remdesivir.
38. The vardenafil for use according to embodiment 35, wherein the antiviral agent is a protease inhibitor.
39. The vardenafil for use according to embodiment 38, wherein the antiviral agent is amprenavir, atazanavir, indinavir, darunavir, fosamprenavir, nelfinavir, ritonavir, saquinavir, tipranavir or lopinavir.
40. The vardenafil for use according to embodiment 35, wherein the antiviral agent is a nucleoside analog.
41. The vardenafil for use according to embodiment 40, wherein the antiviral agent is Remdesivir, ribavirin, didanosine, vidarabine, galidesivir, Remdesivir, emtricitabine, lamivudine, zalcitabine, abacavir, acyclovir, entecavir, stavudine, telbivudine, zidovudine, idoxuridine, or trifluridine.
42. The vardenafil for use according to embodiment 35, wherein the antiviral agent is an interferon.
43. The vardenafil for use according to embodiment 42, wherein the antiviral agent is interferon beta 1b.
44. An antiviral agent for use in combination with vardenafil for the treatment of a coronavirus infection in a subject.
45. The antiviral agent for use according to embodiment 44, wherein the vardenafil and antiviral agent are in a single pharmaceutical composition.
46. The antiviral agent for use according to embodiment 44, wherein the vardenafil and the antiviral agent are in different pharmaceutical compositions.
47. The antiviral agent for use according to any one of embodiments 44 to 46, wherein the antiviral agent targets RNA-dependent RNA polymerase (RdRP).
48. The antiviral agent for use according to any one of embodiments 44 to 47, which is Remdesivir.
49. The antiviral agent for use according to any one of embodiments 44 to 46, wherein the antiviral agent is a protease inhibitor.
50. The antiviral agent for use according to embodiment 49, wherein the antiviral agent is amprenavir, atazanavir, indinavir, darunavir, fosamprenavir, nelfinavir, ritonavir, saquinavir, tipranavir or lopinavir.
51. The antiviral agent for use according to any one of embodiments 44 to 46, wherein the antiviral agent is a nucleoside analog.
52. The antiviral agent for use according to embodiment 51, wherein the antiviral agent is Remdesivir, ribavirin, didanosine, vidarabine, galidesivir, emtricitabine, lamivudine, zalcitabine, abacavir, acyclovir, entecavir, stavudine, telbivudine, zidovudine, idoxuridine, or trifluridine.
53. The antiviral agent for use according to any one of embodiments 44 to 46, wherein the antiviral agent is an interferon.
54. The antiviral agent for use according to embodiment 53, wherein the antiviral agent is interferon beta 1b.
55. A combination comprising vardenafil and an antiviral agent for the treatment of a coronavirus infection in a subject.
56. The combination of embodiment 55, wherein the antiviral agent targets RNA-dependent RNA polymerase (RdRP).
57. The combination of any one of embodiments 55 to 56, wherein the antiviral agent is Remdesivir.
58. The combination of embodiment 57, wherein the antiviral agent is a protease inhibitor.
59. The combination of embodiment 58, wherein the antiviral agent is amprenavir, atazanavir, indinavir, darunavir, fosamprenavir, nelfinavir, ritonavir, saquinavir, tipranavir or lopinavir.
60. The combination of embodiment 57, wherein the antiviral agent is a nucleoside analog.
61. The combination of embodiment 60, wherein the antiviral agent is Remdesivir, ribavirin, didanosine, vidarabine, galidesivir, emtricitabine, lamivudine, zalcitabine, abacavir, acyclovir, entecavir, stavudine, telbivudine, zidovudine, idoxuridine, or trifluridine.
62. The combination of embodiment 57, wherein the antiviral agent is an interferon.
63. The combination of embodiment 62, wherein the antiviral agent is interferon beta 1b.
64. The combination of any one of embodiments 55 to 63, wherein the combination is in the form of a pharmaceutical composition comprising both vardenafil and the antiviral agent.
65. The combination of embodiment 64, wherein the pharmaceutical composition is a solution, tablet, capsule, granule, powder, crystal, gel, liquid, suspension, paste, or pellet.
66. The combination of any one of embodiments 55 to 65, wherein the combination is in the form of a pharmaceutical composition comprising vardenafil and a separate pharmaceutical composition comprising the antiviral agent.
67. The combination of embodiment 66, wherein the pharmaceutical composition comprising vardenafil and the pharmaceutical composition comprising the antiviral agent are each independently a solution, tablet, capsule, granule, powder, crystal, gel, liquid, suspension, paste, or pellet.
68. The combination of embodiment 67, wherein the pharmaceutical composition comprising vardenafil is a tablet and the pharmaceutical composition comprising the antiviral agent is a solution.
69. A method of treating a subject having or suspected of having a coronavirus infection, comprising administering a therapeutically effective amount of vardenafil to the subject.
70. The method of embodiment 69, wherein the subject has a coronavirus infection.
71. The method of embodiment 70, wherein the therapeutically effective amount reduces the severity of one or more symptoms of the coronavirus infection.
72. The method of embodiment 70 or embodiment 71, wherein the therapeutically effective amount reduces the duration of one or more symptoms of the coronavirus infection.
73. The method of any one of embodiments 69 to 72, wherein the therapeutically effective amount reduces the duration of the coronavirus infection.
74. The method of embodiment 69, wherein the subject is suspected of having a coronavirus infection.
75. The method of any one of embodiments 69 to 74, wherein the subject is tested for infection with the coronavirus before and/or after administration of the vardenafil begins.
76. The method of any one of embodiments 69 to 75, which comprises administering the vardenafil to the subject until one, more than one, or all symptoms of the coronavirus infection experienced by the subject are reduced or end.
77. The method of any one of embodiments 69 to 76, which comprises administering the vardenafil to the subject until the subject tests negative for infection with the coronavirus.
78. The method of any one of embodiments 69 to 77, which further comprises testing the subject for infection with the coronavirus.
79. The method of any one of embodiments 69 to 78, wherein the vardenafil is administered via oral, intraperitoneal, intramuscular, intravenous, inhalation, topical, buccal, injection, nasal, transdermal, intradermal, translingual, urethral, vaginal, enteral, rectal, ophthalmic, subcutaneous, irrigation, mouth, throat, transmucosal, intravitreal, otic, perfusion, perivascular, or sublingual administration.
80. The method of any one of embodiments 69 to 78, wherein the vardenafil is administered orally.
81. The method of any one of embodiments 69 to 80, wherein the vardenafil is in a pharmaceutical composition which is a solution, tablet, capsule, granule, powder, crystal, gel, liquid, suspension, paste, or pellet.
82. The method of embodiment 81, wherein the vardenafil is in a pharmaceutical composition which is a tablet.
83. The method of any one of embodiments 69 to 82, wherein the vardenafil is administered as monotherapy.
84. The method of any one of embodiments 69 to 82, wherein the vardenafil is administered in combination with one or more additional agents.
85. The method of embodiment 84, wherein the vardenafil and the one or more additional agents are administered in separate pharmaceutical compositions.
86. The method of embodiment 85, wherein the separate pharmaceutical compositions are each independently administered via oral, intraperitoneal, intramuscular, intravenous, inhalation, topical, buccal, injection, nasal, transdermal, intradermal, translingual, urethral, vaginal, enteral, rectal, ophthalmic, subcutaneous, irrigation, mouth, throat, transmucosal, intravitreal, otic, perfusion, perivascular, or sublingual administration.
87. The method of embodiment 85 or 86, wherein the separate pharmaceutical compositions are each independently a solution, tablet, capsule, granule, powder, crystal, gel, liquid, suspension, paste, or pellet.
88. The method of any one of embodiments 85 to 87, wherein the vardenafil is administered orally and one or more of the one or more additional agents are administered intravenously.
89. The method of embodiment 84, wherein the vardenafil and the one or more additional agents are administered in the same pharmaceutical composition.
90. The method of embodiment 89, wherein the pharmaceutical composition is administered via oral, intraperitoneal, intramuscular, intravenous, inhalation, topical, buccal, injection, nasal, transdermal, intradermal, translingual, urethral, vaginal, enteral, rectal, ophthalmic, subcutaneous, irrigation, mouth, throat, transmucosal, intravitreal, otic, perfusion, perivascular, or sublingual administration.
91. The method of embodiment 89 or 90, wherein the pharmaceutical composition is a solution, tablet, capsule, granule, powder, crystal, gel, liquid, suspension, paste, or pellet.
92. The method of any one of embodiments 84 to 91, wherein the one or more additional agents comprise an antiviral agent.
93. The method of embodiment 92, wherein the antiviral agent targets RNA-dependent RNA polymerase (RdRP).
94. The method of embodiment 92 or embodiment 93, wherein the antiviral agent is Remdesivir.
95. The method of embodiment 92, wherein the antiviral agent is a protease inhibitor.
96. The method of embodiment 95, wherein the antiviral agent is amprenavir, atazanavir, indinavir, darunavir, fosamprenavir, nelfinavir, ritonavir, saquinavir, tipranavir or lopinavir.
97. The method of embodiment 92, wherein the antiviral agent is a nucleoside analog.
98. The method of embodiment 97, wherein the antiviral agent is Remdesivir, ribavirin, didanosine, vidarabine, galidesivir, emtricitabine, lamivudine, zalcitabine, abacavir, acyclovir, entecavir, stavudine, telbivudine, zidovudine, idoxuridine, or trifluridine.
99. The method of embodiment 92, wherein the antiviral agent is an interferon.
100. The method of embodiment 99, wherein the antiviral agent is interferon beta 1b.
101. The method of any one of embodiments 84 to 100, which further comprises administering the one or more additional agents to the subject.
102. The method of any one of embodiments 84 to 101, which comprises administering the one or more additional agents to the subject until one, more than one, or all symptoms of the coronavirus infection experienced by the subject are reduced or end.
103. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, wherein the subject is a human.
104. The use, the vardenafil for use, the antiviral agent for use, the combination, or the method of embodiment 103, wherein the subject is an adult.
105. The use, the vardenafil for use, the antiviral agent for use, the combination, or the method of embodiment 103, wherein the subject is a child.
106. The use, the vardenafil for use, the antiviral agent for use, the combination, or the method of embodiment 105, wherein the child is at least 12 years old.
107. The use, the vardenafil for use, the antiviral agent for use, the combination, or the method of embodiment 105, wherein the child is less than 12 years old.
108. The use, the vardenafil for use, the antiviral agent for use, the combination, or the method of any one of embodiments 103 to 107, wherein the subject weighs at least 40 kg.
109. The use, the vardenafil for use, the antiviral agent for use, the combination, or the method of any one of embodiments 103 to 107, wherein the subject weighs at least 3.5 kg and/or less than 40 kg.
110. The use, vardenafil for use, the antiviral agent for use, the combination, or the method of any one of embodiments 103 to 109, wherein the subject is hospitalized.
111. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 110, wherein the coronavirus is SARS-CoV-2.
112. The use, the vardenafil for use, the antiviral agent for use, the combination, or the method of embodiment 111, wherein the SARS-CoV-2 has a 23403A>G mutation.
113. The use, the vardenafil for use, the antiviral agent for use, the combination, or the method of embodiment 111, wherein the SARS-CoV-2 has a D614G mutation in its spike protein.
114. The use, the vardenafil for use, the antiviral agent for use, the combination, or the method of any one of embodiments 111 to 113, wherein the SARS-CoV-2 has a 14408C>T mutation.
115. The use, the vardenafil for use, the antiviral agent for use, the combination, or the method of any one of embodiments 111 to 113, wherein the SARS-CoV-2 has a P323L mutation in its RdRP protein.
116. The use, the vardenafil for use, the antiviral agent for use, the combination, or the method of any one of embodiments 111 to 115, wherein the SARS-CoV-2 has a 25563G>T mutation.
117. The use, the vardenafil for use, the antiviral agent for use, the combination, or the method of any one of embodiments 111 to 115, wherein the SARS-CoV-2 has a Q57H mutation in its ORF3a protein.
118. The use, the vardenafil for use, the antiviral agent for use, the combination, or the method of any one of embodiments 111 to 117, wherein the SARS-CoV-2 has a 1059C>T mutation.
119. The use, the vardenafil for use, the antiviral agent for use, the combination, or the method of any one of embodiments 111 to 117, wherein the SARS-CoV-2 has a T85I mutation in its NSP2 protein.
120. The use, the vardenafil for use, the antiviral agent for use, the combination, or the method of any one of embodiments 111 to 119, wherein the SARS-CoV-2 has a 28881G>A mutation.
121. The use, the vardenafil for use, the antiviral agent for use, the combination, or the method of any one of embodiments 111 to 119, wherein the SARS-CoV-2 has a R203K mutation in its nucleocapsid.
122. The use, the vardenafil for use, the antiviral agent for use, the combination, or the method of any one of embodiments 111 to 121, wherein the SARS-CoV-2 has a 28883G>C mutation.
123. The use, the vardenafil for use, the antiviral agent for use, the combination, or the method of any one of embodiments 111 to 121, wherein the SARS-CoV-2 has a G204R mutation in its nucleocapsid.
124. The use, the vardenafil for use, the antiviral agent for use, the combination, or the method of any one of embodiments 111 to 123, wherein the SARS-CoV-2 has a 28882G>A mutation.
125. The use, the vardenafil for use, the antiviral agent for use, the combination, or the method of any one of embodiments 111 to 123, wherein the SARS-CoV-2 has a R203K mutation in its nucleocapsid.
126. The use, the vardenafil for use, the antiviral agent for use, the combination, or the method of any one of embodiments 111 to 125, wherein the SARS-CoV-2 has a 28144T>C mutation.
127. The use, the vardenafil for use, the antiviral agent for use, the combination, or the method of any one of embodiments 111 to 125, wherein the SARS-CoV-2 has a L84S mutation in its ORF8 protein.
128. The use, the vardenafil for use, the antiviral agent for use, the combination, or the method of any one of embodiments 111 to 127, wherein the SARS-CoV-2 has a 17858A>G mutation.
129. The use, the vardenafil for use, the antiviral agent for use, the combination, or the method of any one of embodiments 111 to 127, wherein the SARS-CoV-2 has a Y541C mutation in its NSP13 protein.
130. The use, the vardenafil for use, the antiviral agent for use, the combination, or the method of any one of embodiments 111 to 129, wherein the SARS-CoV-2 has a 17747C>T mutation.
131. The use, the vardenafil for use, the antiviral agent for use, the combination, or the method of any one of embodiments 111 to 129, wherein the SARS-CoV-2 has a P504L mutation in its NSP13 protein.
132. The use, the vardenafil for use, the antiviral agent for use, the combination, or the method of any one of embodiments 111 to 131, wherein the SARS-CoV-2 has a 27964C>T mutation.
133. The use, the vardenafil for use, the antiviral agent for use, the combination, or the method of any one of embodiments 111 to 131, wherein the SARS-CoV-2 has a S24L mutation in its ORF8 protein.
134. The use, the vardenafil for use, the antiviral agent for use, the combination, or the method of any one of embodiments 111 to 133, wherein the SARS-CoV-2 has a 3037C>T mutation.
135. The use, the vardenafil for use, the antiviral agent for use, the combination, or the method of any one of embodiments 111 to 134, wherein the SARS-CoV-2 has a 8782C>T mutation.
136. The use, the vardenafil for use, the antiviral agent for use, the combination, or the method of any one of embodiments 111 to 135, wherein the SARS-CoV-2 has a 18060C>T mutation.
137. The use, the vardenafil for use, the antiviral agent for use, the combination, or the method of any one of embodiments 111 to 136, wherein the SARS-CoV-2 has a deletion of amino acids 69-70 (69-70del) in its spike protein.
138. The use, the vardenafil for use, the antiviral agent for use, the combination, or the method of any one of embodiments 111 to 137, wherein the SARS-CoV-2 has a K417N mutation in its spike protein.
139. The use, the vardenafil for use, the antiviral agent for use, the combination, or the method of any one of embodiments 111 to 138, wherein the SARS-CoV-2 has a L452R mutation in its spike protein.
140. The use, the vardenafil for use, the antiviral agent for use, the combination, or the method of any one of embodiments 111 to 139, wherein the SARS-CoV-2 has a Y453F mutation in its spike protein.
141. The use, the vardenafil for use, the antiviral agent for use, the combination, or the method of any one of embodiments 111 to 140, wherein the SARS-CoV-2 has a E484K mutation in its spike protein.
142. The use, the vardenafil for use, the antiviral agent for use, the combination, or the method of any one of embodiments 111 to 141, wherein the SARS-CoV-2 has a N501Y mutation in its spike protein.
143. The use, the vardenafil for use, the antiviral agent for use, the combination, or the method of any one of embodiments 111 to 142, wherein the SARS-CoV-2 has a D614G mutation in its spike protein.
144. The use, the vardenafil for use, the antiviral agent for use, the combination, or the method of any one of embodiments 111 to 143, wherein the SARS-CoV-2 has a P681H mutation in its spike protein.
145. The use, the vardenafil for use, the antiviral agent for use, the combination, or the method of any one of embodiments 111 to 144, wherein the SARS-CoV-2 has a F888L mutation in its spike protein.
146. The use, the vardenafil for use, the antiviral agent for use, the combination, or the method of any one of embodiments 111 to 145, wherein the SARS-CoV-2 has a Q677P mutation in its spike protein.
147. The use, the vardenafil for use, the antiviral agent for use, the combination, or the method of any one of embodiments 111 to 145, wherein the SARS-CoV-2 has a Q677H mutation in its spike protein.
148. The use, the vardenafil for use, the antiviral agent for use, the combination, or the method of embodiment 111, wherein the SARS-CoV-2 is variant B.1.1.7.
149. The use, the vardenafil for use, the antiviral agent for use, the combination, or the method of embodiment 111, wherein the SARS-CoV-2 is variant B.1.351.
150. The use, the vardenafil for use, the antiviral agent for use, the combination, or the method of embodiment 111, wherein the SARS-CoV-2 is variant P.1.
151. The use, the vardenafil for use, the antiviral agent for use, the combination, or the method of embodiment 111, wherein the SARS-CoV-2 is variant P.2.
152. The use, the vardenafil for use, the antiviral agent for use, the combination, or the method of embodiment 111, wherein the SARS-CoV-2 is variant B.1.207.
153. The use, the vardenafil for use, the antiviral agent for use, the combination, or the method of embodiment 111, wherein the SARS-CoV-2 is variant B.1.525.
154. The use, the vardenafil for use, the antiviral agent for use, the combination, or the method of embodiment 111, wherein the SARS-CoV-2 is variant cluster 5.
155. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 110, wherein the coronavirus is SARS-CoV, MERS-CoV, COVID-19, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus, Human coronavirus OC43, Hedgehog coronavirus 1 (EriCoV), Beluga whale coronavirus SW1, Infectious bronchitis virus, Bulbul coronavirus HKU11, or Porcine coronavirus HKU15.
156. The use, the vardenafil for use, the antiviral agent for use, the combination, or the method of embodiment 155, wherein the coronavirus is SARS-CoV.
157. The use, the vardenafil for use, the antiviral agent for use, the combination, or the method of embodiment 155, wherein the coronavirus is MERS-CoV.
158. The use, the vardenafil for use, the antiviral agent for use, the combination, or the method of embodiment 155, wherein the coronavirus is Human coronavirus 229E.
159. The use, the vardenafil for use, the antiviral agent for use, the combination, or the method of embodiment 155, wherein the coronavirus is Human coronavirus NL63.
160. The use, the vardenafil for use, the antiviral agent for use, the combination, or the method of embodiment 155, wherein the coronavirus is Miniopterus bat coronavirus 1.
161. The use, the vardenafil for use, the antiviral agent for use, the combination, or the method of embodiment 155, wherein the coronavirus is Miniopterus bat coronavirus HKU8.
162. The use, the vardenafil for use, the antiviral agent for use, the combination, or the method of embodiment 155, wherein the coronavirus is Porcine epidemic diarrhea virus.
163. The use, the vardenafil for use, the antiviral agent for use, the combination, or the method of embodiment 155, wherein the coronavirus is Rhinolophus bat coronavirus HKU2.
164. The use, the vardenafil for use, the antiviral agent for use, the combination, or the method of embodiment 155, wherein the coronavirus is Scotophilus bat coronavirus 512.
165. The use, the vardenafil for use, the antiviral agent for use, the combination, or the method of embodiment 155, wherein the coronavirus is Betacoronavirus 1.
166. The use, the vardenafil for use, the antiviral agent for use, the combination, or the method of embodiment 155, wherein the coronavirus is Human coronavirus HKU1.
167. The use, the vardenafil for use, the antiviral agent for use, the combination, or the method of embodiment 155, wherein the coronavirus is Murine coronavirus.
168. The use, the vardenafil for use, the antiviral agent for use, the combination, or the method of embodiment 155, wherein the coronavirus is Pipistrellus bat coronavirus HKU5.
169. The use, the vardenafil for use, the antiviral agent for use, the combination, or the method of embodiment 155, wherein the coronavirus is Rousettus bat coronavirus HKU9.
170. The use, the vardenafil for use, the antiviral agent for use, the combination, or the method of embodiment 155, wherein the coronavirus is Severe acute respiratory syndrome-related coronavirus.
171. The use, the vardenafil for use, the antiviral agent for use, the combination, or the method of embodiment 155, wherein the coronavirus is Tylonycteris bat coronavirus HKU4.
172. The use, the vardenafil for use, the antiviral agent for use, the combination, or the method of embodiment 155, wherein the coronavirus is Middle East respiratory syndrome-related coronavirus.
173. The use, the vardenafil for use, the antiviral agent for use, the combination, or the method of embodiment 155, wherein the coronavirus is Human coronavirus OC43. Hedgehog coronavirus 1 (EriCoV).
174. The use, the vardenafil for use, the antiviral agent for use, the combination, or the method of embodiment 155, wherein the coronavirus is Beluga whale coronavirus SW1.
175. The use, the vardenafil for use, the antiviral agent for use, the combination, or the method of embodiment 155, wherein the coronavirus is Infectious bronchitis virus.
176. The use, the vardenafil for use, the antiviral agent for use, the combination, or the method of embodiment 155, wherein the coronavirus is Bulbul coronavirus HKU11.
177. The use, the vardenafil for use, the antiviral agent for use, the combination, or the method of embodiment 155, wherein the coronavirus is Porcine coronavirus HKU15.
178. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 110, wherein the coronavirus is:
   a. Alphacoronavirus, which is optionally Colacovirus or Bat coronavirus CDPHE15;
   b. Decacovirus, which is optionally Bat coronavirus HKU10 or Rhinolophus ferrumequinum alphacoronavirus HuB-2013;
   c. Duvinacovirus, which is optionally Human coronavirus 229E;
   d. Luchacovirus, which is optionally Lucheng Rn rat coronavirus;
   e. Minacovirus, which is optionally Ferret coronavirus or Mink coronavirus 1;
   f. Minunacovirus, which is optionally Miniopterus bat coronavirus 1 or Miniopterus bat coronavirus HKU8;
   g. Myotacovirus, which is optionally Myotis ricketti alphacoronavirus Sax-2011;
   h. Nyctacovirus, which is optionally Nyctalus velutinus alphacoronavirus SC-2013;
   i. Pedacovirus, which is optionally Porcine epidemic diarrhea virus or Scotophilus bat coronavirus 512;
   j. Rhinacovirus, which is optionally Rhinolophus bat coronavirus HKU2;
   k. Setracovirus, which is optionally Human coronavirus NL63 or NL63-related bat coronavirus strain BtKYNL63-9b;
   l. Tegacovirus, which is optionally Alphacoronavirus 1 - type species;
   m. Betacoronavirus, which is optionally Embecovirus, Betacoronavirus 1, Human coronavirus OC43, China Rattus coronavirus HKU24, Human coronavirus HKU1, or Murine coronavirus - type species;
   n. Hibecovirus, which is optionally Bat Hp-betacoronavirus Zhejiang2013;
   o. Merbecovirus, which is optionally Hedgehog coronavirus 1, Middle East respiratory syndrome-related coronavirus (MERS-CoV), Pipistrellus bat coronavirus HKU5, or Tylonycteris bat coronavirus HKU4;
   p. Nobecovirus, which is optionally Rousettus bat coronavirus GCCDC1 or Rousettus bat coronavirus HKU9;
   q. Sarbecovirus, which is optionally Severe acute respiratory syndrome-related coronavirus, Severe acute respiratory syndrome coronavirus (SARS-CoV), or COVID-19 (Severe acute respiratory syndrome coronavirus 2 (SARS-CoV-2,Wuhan 2020 nCOV));
   r. Deltacoronavirus, which is optionally Andecovirus or Wigeon coronavirus HKU20;
   s. Buldecovirus, which is optionally Bulbul coronavirus HKU11 – type species, Porcine coronavirus HKU15, Munia coronavirus HKU13, or White-eye coronavirus HKU16;
   t. Herdecovirus, which is optionally Night heron coronavirus HKU19;
   u. Moordecovirus, which is optionally Common moorhen coronavirus HKU21;
   v. Gammacoronavirus, which is optionally Cegacovirus, Beluga whale coronavirus SW1, or Igacovirus; or
   w. Avian coronavirus-type species.
179. The use, the vardenafil for use, the antiviral agent for use, the combination, or the method of embodiment 178, wherein the coronavirus is Alphacoronavirus.
180. The use, the vardenafil for use, the antiviral agent for use, the combination, or the method of embodiment 178, wherein the coronavirus is Colacovirus
181. The use, the vardenafil for use, the antiviral agent for use, the combination, or the method of embodiment 178, wherein the coronavirus is Bat coronavirus CDPHE15.
182. The use, the vardenafil for use, the antiviral agent for use, the combination, or the method of embodiment 178, wherein the coronavirus is Decacovirus.
183. The use, the vardenafil for use, the antiviral agent for use, the combination, or the method of embodiment 178, wherein the coronavirus is Bat coronavirus HKU10.
184. The use, the vardenafil for use, the antiviral agent for use, the combination, or the method of embodiment 178, wherein the coronavirus is Rhinolophus ferrumequinum alphacoronavirus HuB-2013.
185. The use, the vardenafil for use, the antiviral agent for use, the combination, or the method of embodiment 178, wherein the coronavirus is Duvinacovirus.
186. The use, the vardenafil for use, the antiviral agent for use, the combination, or the method of embodiment 178, wherein the coronavirus is Human coronavirus 229E.
187. The use, the vardenafil for use, the antiviral agent for use, the combination, or the method of embodiment 178, wherein the coronavirus is Luchacovirus.
188. The use, the vardenafil for use, the antiviral agent for use, the combination, or the method of embodiment 178, wherein the coronavirus is Lucheng Rn rat coronavirus.
189. The use, the vardenafil for use, the antiviral agent for use, the combination, or the method of embodiment 178, wherein the coronavirus is Minacovirus.
190. The use, the vardenafil for use, the antiviral agent for use, the combination, or the method of embodiment 178, wherein the coronavirus is Ferret coronavirus.
191. The use, the vardenafil for use, the antiviral agent for use, the combination, or the method of embodiment 178, wherein the coronavirus is Mink coronavirus 1.
192. The use, the vardenafil for use, the antiviral agent for use, the combination, or the method of embodiment 178, wherein the coronavirus is Minunacovirus.
193. The use, the vardenafil for use, the antiviral agent for use, the combination, or the method of embodiment 178, wherein the coronavirus is Miniopterus bat coronavirus 1.
194. The use, the vardenafil for use, the antiviral agent for use, the combination, or the method of embodiment 178, wherein the coronavirus is Miniopterus bat coronavirus HKU8.
195. The use, the vardenafil for use, the antiviral agent for use, the combination, or the method of embodiment 178, wherein the coronavirus is Myotacovirus.
196. The use, the vardenafil for use, the antiviral agent for use, the combination, or the method of embodiment 178, wherein the coronavirus is Myotis ricketti alphacoronavirus Sax-2011.
197. The use, the vardenafil for use, the antiviral agent for use, the combination, or the method of embodiment 178, wherein the coronavirus is Nyctacovirus.
198. The use, the vardenafil for use, the antiviral agent for use, the combination, or the method of embodiment 178, wherein the coronavirus is Nyctalus velutinus alphacoronavirus SC-2013.
199. The use, the vardenafil for use, the antiviral agent for use, the combination, or the method of embodiment 178, wherein the coronavirus is Pedacovirus.
200. The use, the vardenafil for use, the antiviral agent for use, the combination, or the method of embodiment 178, wherein the coronavirus is Porcine epidemic diarrhea virus.
201. The use, the vardenafil for use, the antiviral agent for use, the combination, or the method of embodiment 178, wherein the coronavirus is Scotophilus bat coronavirus 512.
202. The use, the vardenafil for use, the antiviral agent for use, the combination, or the method of embodiment 178, wherein the coronavirus is Rhinacovirus.
203. The use, the vardenafil for use, the antiviral agent for use, the combination, or the method of embodiment 178, wherein the coronavirus is Rhinolophus bat coronavirus HKU2.
204. The use, the vardenafil for use, the antiviral agent for use, the combination, or the method of embodiment 178, wherein the coronavirus is Setracovirus.
205. The use, the vardenafil for use, the antiviral agent for use, the combination, or the method of embodiment 178, wherein the coronavirus is Human coronavirus NL63.
206. The use, the vardenafil for use, the antiviral agent for use, the combination, or the method of embodiment 178, wherein the coronavirus is NL63-related bat coronavirus strain BtKYNL63-9b.
207. The use, the vardenafil for use, the antiviral agent for use, the combination, or the method of embodiment 178, wherein the coronavirus is Tegacovirus.
208. The use, the vardenafil for use, the antiviral agent for use, the combination, or the method of embodiment 178, wherein the coronavirus is Alphacoronavirus 1 – type species.
209. The use, the vardenafil for use, the antiviral agent for use, the combination, or the method of embodiment 178, wherein the coronavirus is Betacoronavirus.
210. The use, the vardenafil for use, the antiviral agent for use, the combination, or the method of embodiment 178, wherein the coronavirus is Embecovirus.
211. The use, the vardenafil for use, the antiviral agent for use, the combination, or the method of embodiment 178, wherein the coronavirus is Betacoronavirus 1.
212. The use, the vardenafil for use, the antiviral agent for use, the combination, or the method of embodiment 178, wherein the coronavirus is Human coronavirus OC43.
213. The use, the vardenafil for use, the antiviral agent for use, the combination, or the method of embodiment 178, wherein the coronavirus is China Rattus coronavirus HKU24.
214. The use, the vardenafil for use, the antiviral agent for use, the combination, or the method of embodiment 178, wherein the coronavirus is Human coronavirus HKU1.
215. The use, the vardenafil for use, the antiviral agent for use, the combination, or the method of embodiment 178, wherein the coronavirus is Murine coronavirus – type species.
216. The use, the vardenafil for use, the antiviral agent for use, the combination, or the method of embodiment 178, wherein the coronavirus is Hibecovirus.
217. The use, the vardenafil for use, the antiviral agent for use, the combination, or the method of embodiment 178, wherein the coronavirus is Bat Hp-betacoronavirus Zhejiang2013.
218. The use, the vardenafil for use, the antiviral agent for use, the combination, or the method of embodiment 178, wherein the coronavirus is Merbecovirus.
219. The use, the vardenafil for use, the antiviral agent for use, the combination, or the method of embodiment 178, wherein the coronavirus is Hedgehog coronavirus 1.
220. The use, the vardenafil for use, the antiviral agent for use, the combination, or the method of embodiment 178, wherein the coronavirus is Middle East respiratory syndrome-related coronavirus (MERS-CoV).
221. The use, the vardenafil for use, the antiviral agent for use, the combination, or the method of embodiment 178, wherein the coronavirus is Pipistrellus bat coronavirus HKU5.
222. The use, the vardenafil for use, the antiviral agent for use, the combination, or the method of embodiment 178, wherein the coronavirus is Tylonycteris bat coronavirus HKU4.
223. The use, the vardenafil for use, the antiviral agent for use, the combination, or the method of embodiment 178, wherein the coronavirus is Nobecovirus.
224. The use, the vardenafil for use, the antiviral agent for use, the combination, or the method of embodiment 178, wherein the coronavirus is Rousettus bat coronavirus GCCDC1.
225. The use, the vardenafil for use, the antiviral agent for use, the combination, or the method of embodiment 178, wherein the coronavirus is Rousettus bat coronavirus HKU9.
226. The use, the vardenafil for use, the antiviral agent for use, the combination, or the method of embodiment 178, wherein the coronavirus is Sarbecovirus.
227. The use, the vardenafil for use, the antiviral agent for use, the combination, or the method of embodiment 178, wherein the coronavirus is Severe acute respiratory syndrome-related coronavirus.
228. The use, the vardenafil for use, the antiviral agent for use, the combination, or the method of embodiment 178, wherein the coronavirus is Severe acute respiratory syndrome coronavirus (SARS-CoV).
229. The use, the vardenafil for use, the antiviral agent for use, the combination, or the method of embodiment 178, wherein the coronavirus is Deltacoronavirus.
230. The use, the vardenafil for use, the antiviral agent for use, the combination, or the method of embodiment 178, wherein the coronavirus is Andecovirus.
231. The use, the vardenafil for use, the antiviral agent for use, the combination, or the method of embodiment 178, wherein the coronavirus is Wigeon coronavirus HKU20.
232. The use, the vardenafil for use, the antiviral agent for use, the combination, or the method of embodiment 178, wherein the coronavirus is Buldecovirus.
233. The use, the vardenafil for use, the antiviral agent for use, the combination, or the method of embodiment 178, wherein the coronavirus is Bulbul coronavirus HKU11 -type species.
234. The use, the vardenafil for use, the antiviral agent for use, the combination, or the method of embodiment 178, wherein the coronavirus is Porcine coronavirus HKU15.
235. The use, the vardenafil for use, the antiviral agent for use, the combination, or the method of embodiment 178, wherein the coronavirus is Munia coronavirus HKU13.
236. The use, the vardenafil for use, the antiviral agent for use, the combination, or the method of embodiment 178, wherein the coronavirus is White-eye coronavirus HKU16.
237. The use, the vardenafil for use, the antiviral agent for use, the combination, or the method of embodiment 178, wherein the coronavirus is Herdecovirus.
238. The use, the vardenafil for use, the antiviral agent for use, the combination, or the method of embodiment 178, wherein the coronavirus is Night heron coronavirus HKU19.
239. The use, the vardenafil for use, the antiviral agent for use, the combination, or the method of embodiment 178, wherein the coronavirus is Moordecovirus.
240. The use, the vardenafil for use, the antiviral agent for use, the combination, or the method of embodiment 178, wherein the coronavirus is Common moorhen coronavirus HKU21.
241. The use, the vardenafil for use, the antiviral agent for use, the combination, or the method of embodiment 178, wherein the coronavirus is Gammacoronavirus.
242. The use, the vardenafil for use, the antiviral agent for use, the combination, or the method of embodiment 178, wherein the coronavirus is Cegacovirus.
243. The use, the vardenafil for use, the antiviral agent for use, the combination, or the method of embodiment 178, wherein the coronavirus is Beluga whale coronavirus SW1.
244. The use, the vardenafil for use, the antiviral agent for use, the combination, or the method of embodiment 178, wherein the coronavirus is Igacovirus.
245. The use, the vardenafil for use, the antiviral agent for use, the combination, or the method of embodiment 178, wherein the coronavirus is Avian coronavirus-type species.
246. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 245, wherein the vardenafil is in the form of a pharmaceutically acceptable salt.
247. The use, the vardenafil for use, the antiviral agent for use, the combination, or the method of embodiment 246, wherein the pharmaceutically acceptable salt is an ammonium salt, hydrochloride, carbonate, bicarbonate, acetate, lactate, butyrate, propionate, sulfate, methanesulfonate, citrates, tartrate, nitrate, sulfonate, oxalate, succinate, or combination thereof.
248. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 247, wherein the vardenafil is in the form of a solvate.
249. The use, the vardenafil for use, the antiviral agent for use, the combination, or the method of embodiment 248, wherein the solvate is a hydrate.
250. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 245, wherein the vardenafil is in the form of vardenafil hydrochloride.
251. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 245, wherein the vardenafil is in the form of vardenafil hydrochloride trihydrate.
252. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 251, wherein the vardenafil is in amorphous form.
253. The use according to any one of embodiments 1 to 27, the vardenafil for use according to any one of embodiments 28 to 43, the antiviral agent for use according to any one of embodiments 44 to 54, the combination of any one of embodiments 55 to 68, or the method of any one of embodiments 69 to 102, or the vardenafil for use, the antiviral agent for use, the combination, or the method of any one of embodiments 103 to 251, wherein the vardenafil is in a crystalline form.

While various specific embodiments have been illustrated and described, it will be appreciated that various changes can be made without departing from the spirit and scope of the disclosure(s).

### 8. Citation of References

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. Use of vardenafil for the manufacture of a medicament for treating a coronavirus infection in a subject.

2. The use according to claim 1, wherein the medicament is for administration as monotherapy.

3. The use according to claim 1, wherein the medicament is for administration in combination with one or more additional agents, and wherein the one or more additional agents comprise an antiviral agent.

4. The use according to claim 3, wherein the antiviral agent targets RNA-dependent RNA polymerase (RdRP).

5. The use according to claim 4, wherein the antiviral agent is Remdesivir.

6. The use according to claim 1, wherein the subject is a human.

7. The method of claim 1, wherein the coronavirus is selected from Colacovirus; Bat coronavirus CDPHE15; Bat coronavirus HKU10; Rhinolophus ferrumequinum alphacoronavirus HuB-2013; Human coronavirus 229E; Lucheng Rn rat coronavirus; Ferret coronavirus; Mink coronavirus 1; Miniopterus bat coronavirus 1; Miniopterus bat coronavirus HKU8; Myotis ricketti alphacoronavirus Sax-2011; Nyctalus velutinus alphacoronavirus SC-2013; Porcine epidemic diarrhea virus; Scotophilus bat coronavirus 512; Rhinolophus bat coronavirus HKU2; Human coronavirus NL63; NL63-related bat coronavirus strain BtKYNL63-9b; Alphacoronavirus 1 – type species; Embecovirus, Betacoronavirus 1, Human coronavirus OC43, China Rattus coronavirus HKU24, Human coronavirus HKU1; Murine coronavirus - type species; Bat Hp-betacoronavirus Zhejiang2013; Hedgehog coronavirus 1, Middle East respiratory syndrome-related coronavirus (MERS-CoV), Pipistrellus bat coronavirus HKU5; Tylonycteris bat coronavirus HKU4; Rousettus bat coronavirus GCCDC1; Rousettus bat coronavirus HKU9 Severe acute respiratory syndrome-related coronavirus, Severe acute respiratory syndrome coronavirus (SARS-CoV); SARS-CoV-2; Andecovirus; Wigeon coronavirus HKU20; Bulbul coronavirus HKU11 - type species; Porcine coronavirus HKU15; Munia coronavirus HKU13; White-eye coronavirus HKU16; Night heron coronavirus HKU19; Common moorhen coronavirus HKU21; Cegacovirus; Beluga whale coronavirus SW1; Igacovirus; or Avian coronavirus-type species.

8. The use according to claim 6, wherein the coronavirus is SARS-CoV-2 and wherein the SARS-CoV-2 has one of more of the following mutations: a D614G mutation in its spike protein; a P323L mutation in its RdRP protein; a Q57H mutation in its ORF3a protein; a T85I mutation in its NSP2 protein; a R203K mutation in its nucleocapsid; a G204R mutation in its nucleocapsid; a L84S mutation in its ORF8 protein; a Y541C mutation in its NSP13 protein; a P504L mutation in its NSP13 protein; a S24L mutation in its ORF8 protein; a deletion of amino acids 69-70 (69-70del) in its spike protein; a K417N mutation in its spike protein; a L452R mutation in its spike protein; a Y453F mutation in its spike protein; a E484K mutation in its spike protein; a N501Y mutation in its spike protein; a D614G mutation in its spike protein; a P681H mutation in its spike protein; a F888L mutation in its spike protein; or a Q677P or Q677H mutation in its spike protein.

9. The use according to claim 1, wherein the coronavirus is SARS-CoV-2 and wherein the SARS-CoV-2 is variant B.1.1.7; B.1.351; P.1; P.2; B.1.207; B.1.525; or variant cluster 5.

10. The use according to claim 1, wherein the vardenafil is in the form of vardenafil hydrochloride.

11. The use according to claim 1, wherein the vardenafil is in the form of vardenafil hydrochloride trihydrate.

12. A composition comprising a combination of vardenafil and an antiviral agent for the treatment of a coronavirus infection in a subject.

13. The composition of claim 12, wherein the antiviral agent targets RNA-dependent RNA polymerase (RdRP).

14. The composition of claim 12, wherein the antiviral agent is Remdesivir.

15. The composition of claim 12, wherein the subject is a human.

16. The composition of claim 12, wherein the coronavirus is Colacovirus; Bat coronavirus CDPHE15; Bat coronavirus HKU10; Rhinolophus ferrumequinum alphacoronavirus HuB-2013; Human coronavirus 229E; Lucheng Rn rat coronavirus; Ferret coronavirus; Mink coronavirus 1; Miniopterus bat coronavirus 1; Miniopterus bat coronavirus HKU8; Myotis ricketti alphacoronavirus Sax-2011; Nyctalus velutinus alphacoronavirus SC-2013; Porcine epidemic diarrhea virus; Scotophilus bat coronavirus 512; Rhinolophus bat coronavirus HKU2; Human coronavirus NL63; NL63-related bat coronavirus strain BtKYNL63-9b; Alphacoronavirus 1 – type species; Embecovirus, Betacoronavirus 1, Human coronavirus OC43, China Rattus coronavirus HKU24, Human coronavirus HKU1; Murine coronavirus - type species; Bat Hp-betacoronavirus Zhejiang2013; Hedgehog coronavirus 1, Middle East respiratory syndrome-related coronavirus (MERS-CoV), Pipistrellus bat coronavirus HKU5; Tylonycteris bat coronavirus HKU4; Rousettus bat coronavirus GCCDC1; Rousettus bat coronavirus HKU9 Severe acute respiratory syndrome-related coronavirus, Severe acute respiratory syndrome coronavirus (SARS-CoV); SARS-CoV-2; Andecovirus; Wigeon coronavirus HKU20; Bulbul coronavirus HKU11 – type species; Porcine coronavirus HKU15; Munia coronavirus HKU13; White-eye coronavirus HKU16; Night heron coronavirus HKU19; Common moorhen coronavirus HKU21; Cegacovirus; Beluga whale coronavirus SW1; Igacovirus; or Avian coronavirus-type species.

17. The composition of claim 15, wherein the coronavirus is SARS-CoV-2 and wherein the SARS-CoV-2 has one of more of the following mutations: a D614G mutation in its spike protein; a P323L mutation in its RdRP protein; a Q57H mutation in its ORF3a protein; a T85I mutation in its NSP2 protein; a R203K mutation in its nucleocapsid; a G204R mutation in its nucleocapsid; a L84S mutation in its ORF8 protein; a Y541C mutation in its NSP13 protein; a P504L mutation in its NSP13 protein; a S24L mutation in its ORF8 protein; a deletion of amino acids 69-70 (69-70del) in its spike protein; a K417N mutation in its spike protein; a L452R mutation in its spike protein; a Y453F mutation in its spike protein; a E484K mutation in its spike protein; a N501Y mutation in its spike protein; a D614G mutation in its spike protein; a P681H mutation in its spike protein; a F888L mutation in its spike protein; a Q677P or Q677H mutation in its spike protein.

18. The composition of claim 12, wherein the coronavirus is SARS-CoV-2 and wherein the SARS-CoV-2 is variant B.1.1.7;B.1.351; P.1; P.2; B.1.207; B.1.525; or variant cluster 5.

19. The composition of claim 12, wherein the vardenafil is in the form of vardenafil hydrochloride.

20. The composition of claim 12, wherein the vardenafil is in the form of vardenafil hydrochloride trihydrate.
